(19) United States
(12) Patent Application Publication
(10) Pub. No.: US 2026/0098035 A1
(43) Pub. Date: Apr. 9, 2026

(54) SALTS AND SOLID FORMS OF (S)- OR RACEMIC 3-(4-((4-(MORPHOLINOMETHYL)BENZYL)OXY)-1-OXOISOINDOLIN-2-YL)PIPERIDINE-2,6-DIONE AND METHODS OF USING THE SAME

(71) Applicant: CELGENE CORPORATION, Summit, NJ (US)

(72) Inventors: Lianfeng HUANG, Basking Ridge, NJ (US); Daozhong ZOU, Raritan, NJ (US); Tsz Chung LAI, Cedar Grove, NJ (US)

(21) Appl. No.: 19/113,397

(22) PCT Filed: Sep. 19, 2023
(86) PCT No.: PCT/US2023/074523

§ 371 Date: Mar. 19, 2025

Related U.S. Application Data
(60) Provisional application No. 63/408,358, filed on Sep. 20, 2022.

(51) Int. Cl. C07D 413/14; A61K 31/5377; A61K 31/573; C07C 55/10; C07C 59/06; C07C 59/08; C07C 59/265; C07C 59/50; C07K 16/28

(52) U.S. Cl. CPC C07D 413/14; A61K 31/5377; A61K 31/573; C07C 55/10; C07C 59/06; C07C 59/08; C07C 59/265; C07C 59/50; C07K 16/2896; C07B 2200/13

(57) ABSTRACT

Provided herein are salts of and solid forms comprising free base or salts of (S)- or racemic 3-(4-((4-(morpholinomethyl)benzyl)oxy)-1-oxoisoindolin-2-yl)piperidine-2,6-dione. Pharmaceutical compositions comprising such salts and solid forms and methods of use of such salts and solid forms for treating, preventing, and managing various disorders are also provided herein.

### CROSS-REFERENCE TO RELATED APPLICATIONS

This application claims priority to U.S. Provisional Application No. 63/408,358, filed on Sep. 20, 2022, the entirety of which is incorporated herein by reference.

### FIELD

Provided herein are salts of and solid forms comprising free base or salts of(S)- or racemic 3-(4-((4-(morpholinomethyl)benzyl)oxy)-1-oxoisoindolin-2-yl) piperidine-2,6-dione. Pharmaceutical compositions comprising such salts and solid forms and methods of use of such salts and solid forms for treating, preventing, and managing various disorders are also provided herein.

### BACKGROUND

Alternative solid forms of pharmaceutical compounds have emerged as a possible approach to modulate or enhance the physical and chemical properties of drug products. The identification and selection of a solid form of a pharmaceutical compound are complex, given that a change in solid form may affect a variety of physical and chemical properties, which may provide benefits or drawbacks in processing, formulation, stability, bioavailability, storage, handling (e.g., shipping), among other important pharmaceutical characteristics. Useful pharmaceutical solid forms include crystalline solids and amorphous solids, depending on the product and its mode of administration. Amorphous solids are characterized by a lack of long-range structural order, whereas crystalline solids are characterized by structural periodicity. The desired class of pharmaceutical solid depends upon the specific application; amorphous solids are sometimes selected on the basis of, e.g., an enhanced dissolution profile, while crystalline solids may be desirable for properties such as, e.g., physical or chemical stability (see, e.g., S. R. Vippagunta et al., Adv. Drug. Deliv. Rev., (2001) 48:3-26; L. Yu, Adv. Drug. Deliv. Rev., (2001) 48:27-42).

Notably, it is not possible to predict a priori if crystalline forms of a compound even exist, let alone how to successfully prepare them (see, e.g., Braga and Grepioni, 2005, “Making crystals from crystals: a green route to crystal engineering and polymorphism,” Chem. Commun.: 3635-3645 (with respect to crystal engineering, if instructions are not very precise and/or if other external factors affect the process, the result can be unpredictable); Jones et al., 2006, Pharmaceutical Cocrystals: An Emerging Approach to Physical Property Enhancement,” MRS Bulletin 31:875-879 (at present it is not generally possible to computationally predict the number of observable polymorphs of even the simplest molecules); Price, 2004, “The computational prediction of pharmaceutical crystal structures and polymorphism,” Advanced Drug Delivery Reviews 56:301-319 (“Price”); and Bernstein, 2004, “Crystal Structure Prediction and Polymorphism,” ACA Transactions 39:14-23 (a great deal still needs to be learned and done before one can state with any degree of confidence the ability to predict a crystal structure, much less polymorphic forms)).

The type of salt form of a particular active pharmaceutical ingredient may affect certain properties of the active pharmaceutical ingredient. These properties include solubility, stability, and bioavailability.

The variety of possible solid forms, including both free base forms and salt forms, creates potential diversity in physical and chemical properties for a given pharmaceutical compound. The discovery and selection of solid forms are of great importance in the development of an effective, stable and marketable pharmaceutical product.

### SUMMARY

Provided herein are solid forms (e.g., crystalline forms, amorphous forms, polymorphs or mixtures thereof) comprising Compound 1:

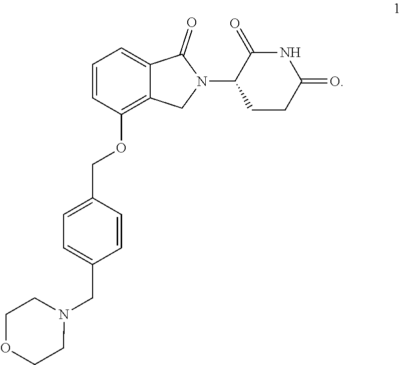 [SMILES: O=C1CC[C@H](N2Cc3c(OCc4ccc(CN5CCOCC5)cc4)cccc3C2=O)C(=O)N1]

In one embodiment, the solid form comprises a free base of Compound 1. In one embodiment, the solid form is Form A, Form B, Form C, or Form D of a free base of Compound 1, as provided herein.

In one embodiment, the solid form comprises a salt of Compound 1.

In one embodiment, the solid form comprises a hydrobromide salt of Compound 1. In one embodiment, the solid form is Form A of a hydrobromide salt of Compound 1, as provided herein.

In one embodiment, the solid form comprises a tosylate salt of Compound 1. In one embodiment, the solid form is Form A of a tosylate salt of Compound 1, as provided herein.

In one embodiment, the solid form comprises a DL-mandelate salt of Compound 1. In one embodiment, the solid form is Form A of a DL-mandelate salt of Compound 1, as provided herein.

In one embodiment, the solid form comprises a glycolate salt of Compound 1. In one embodiment, the solid form is Form A of a glycolate salt of Compound 1, as provided herein.

In one embodiment, the solid form comprises a succinate salt of Compound 1. In one embodiment, the solid form is Form A of a succinate salt of Compound 1, as provided herein.

In one embodiment, the solid form comprises a L-lactate salt of Compound 1. In one embodiment, the solid form is Form A of a L-lactate salt of Compound 1, as provided herein.

In one embodiment, the solid form comprises a citrate salt of Compound 1. In one embodiment, the solid form is Form A of a citrate salt of Compound 1, as provided herein.

Also provided herein are salts of Compound 1. In one embodiment, the salt is a hydrobromide salt, a tosylate salt, a DL-mandelate salt, a glycolate salt, a succinate salt, a L-lactate salt, or a citrate salt.

Provided herein are solid forms (e.g., crystalline forms, amorphous forms, polymorphs or mixtures thereof) comprising Compound 2:

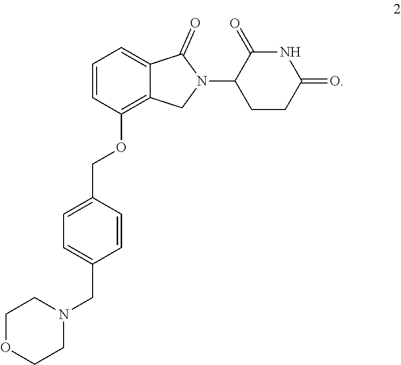 [SMILES: O=C1CCC(N2Cc3c(OCc4ccc(CN5CCOCC5)cc4)cccc3C2=O)C(=O)N1]

In one embodiment, the solid form comprises a free base of Compound 2. In one embodiment, the solid form is Form A of a free base of Compound 2, as provided herein.

In one embodiment, the solid form comprises a salt of Compound 2. In one embodiment, the solid form comprises a hydrochloride salt of Compound 2. In one embodiment, the solid form is Form A or Form B of a hydrochloride salt of Compound 1, as provided herein.

Also provided herein are salts of Compound 2. In one embodiment, the salt is a hydrochloride salt.

Also provided herein are methods of preparing, isolating, and characterizing the solid forms.

The solid forms provided may be characterized using a number of methods known to a person skilled in the art, including, but not limited to, single crystal X-ray diffraction, X-ray powder diffraction (PXRD), microscopy (e.g., optical microscopy, scanning electron microscopy (SEM)), thermal analysis (e.g., differential scanning calorimetry (DSC), thermal gravimetric analysis (TGA), and hot-stage microscopy), dynamic vapor sorption (DVS), spectroscopy (e.g., infrared, Raman, and nuclear magnetic resonance), high performance liquid chromatography (HPLC). The particle size and size distribution of the solid form provided herein may be determined by conventional methods, such as laser light scattering technique.

The purity of the solid forms and salts provided herein may be determined by standard analytical methods, such as thin layer chromatography (TLC), gel electrophoresis, gas chromatography, high performance liquid chromatography (HPLC), and mass spectrometry.

While not intending to be bound by any particular theory, certain solid forms and salts are characterized by physical properties, e.g., stability, solubility and dissolution rate, appropriate for pharmaceutical and therapeutic dosage forms. Moreover, while not wishing to be bound by any particular theory, certain solid forms and salts are characterized by physical properties (e.g., density, compressibility, hardness, morphology, cleavage, stickiness, solubility, water uptake, electrical properties, thermal behavior, solid-state reactivity, physical stability, and chemical stability) affecting particular processes (e.g., yield, filtration, washing, drying, milling, mixing, tableting, flowability, dissolution, formulation, and lyophilization) which make certain solid forms and salts suitable for the manufacture of a solid dosage form. Such properties can be determined using particular analytical chemical techniques, including solid-state analytical techniques (e.g., X-ray diffraction, microscopy, spectroscopy and thermal analysis), as described herein and known in the art. While not intending to be bound by any particular theory, certain solid forms and salts provided herein exhibit suitable pharmaceutical properties, e.g., pharmaceutical kinetics, pharmaceutical dynamics, half-life, Cₘₐₓ, and bioavailability. Such properties can be determined using assays known to the skilled artisan.

The solid forms provided herein are useful as active pharmaceutical ingredients for the preparation of formulations for use in animals or humans. Thus, embodiments herein encompass the use of these solid forms as a final drug substance. One embodiment provide solid forms useful in making final dosage forms with improved properties, e.g., powder flow properties, compaction properties, tableting properties, stability properties, and excipient compatibility properties, among others, that are needed for manufacturing, processing, formulation and/or storage of final drug products. One embodiment herein provide pharmaceutical compositions comprising a single-component crystal form, a multiple-component crystal form, a single-component amorphous form and/or a multiple-component amorphous form comprising Compound 1 and a pharmaceutically acceptable diluent, excipient or carrier.

Also provided are pharmaceutical compositions formulated for administration by an appropriate route and means containing effective concentrations of a solid form comprising Compound 1 or Compound 2 provided herein, and optionally comprising at least one pharmaceutical carrier.

Also provided herein are methods of using a solid form comprising Compound 1 or Compound 2 provided herein for treating, preventing or managing cancer or an immune-related or inflammatory disease. In one embodiment, the method is for treating the cancer or the disease. In one embodiment, the method is for preventing the cancer or the disease. In one embodiment, the method is for managing the cancer or the disease.

In one embodiment, the cancer is lymphoma, leukemia, or myeloma. In one embodiment, the cancer is multiple myeloma (MM), non-Hodgkin's lymphoma (NHL), Hodgkin's lymphoma (HL), or glioblastoma. In one embodiment, the disease is systemic lupus erythematosus, cutaneous lupus erythematosus, or scleroderma.

These and other aspects of the subject matter described herein will become evident upon reference to the following detailed description.

### BRIEF DESCRIPTION OF THE DRAWINGS

FIG. 1 provides a representative X-ray powder diffraction (XRPD) pattern of Form A of a free base of Compound 1.

FIG. 2 provides a representative XRPD pattern of Form B of a free base of Compound 1.

FIG. 3 provides a representative XRPD pattern of Form C of a free base of Compound 1.

FIG. 4 provides a representative XRPD pattern of Form D of a free base of Compound 1.

FIG. 5 provides a representative differential scanning calorimetry (DSC) thermogram of Form D of a free base of Compound 1.

FIG. 6 provides a representative XRPD pattern of Form A of a hydrobromide salt of Compound 1.

FIG. 7 provides a representative XRPD pattern of Form A of a tosylate salt of Compound 1.

FIG. 8 provides a representative DSC thermogram of Form A of a tosylate salt of Compound 1.

FIG. 9 provides a representative thermal gravimetric analysis (TGA) thermogram of Form A of a tosylate salt of Compound 1.

FIG. 10 provides a representative XRPD pattern of Form A of a DL-mandelate salt of Compound 1.

FIG. 11 provides a representative XRPD pattern of Form A of a glycolate salt of Compound 1.

FIG. 12 provides a representative XRPD pattern of Form A of a succinate salt of Compound 1.

FIG. 13 provides a representative XRPD pattern of Form A of a L-lactate salt of Compound 1.

FIG. 14 provides a representative XRPD pattern of Form A of a citrate salt of Compound 1.

FIG. 15 provides a representative XRPD pattern of Form A of a free base of Compound 2.

FIG. 16 provides a representative XRPD pattern of Form A of a hydrochloride salt of Compound 2.

FIG. 17 provides a representative DSC thermogram of Form A of a hydrochloride salt of Compound 2.

FIG. 18 provides a representative TGA thermogram of Form A of a hydrochloride salt of Compound 2.

FIG. 19 provides a representative XRPD pattern of Form B of a hydrochloride salt of Compound 2.

### DETAILED DESCRIPTION

### Definitions

Unless defined otherwise, all technical and scientific terms used herein have the same meaning as is commonly understood by one of ordinary skill in the art. All patents, applications, published applications and other publications are incorporated by reference in their entirety. In the event that there is a plurality of definitions for a term herein, those in this section prevail unless stated otherwise.

As used herein, and in the specification and the accompanying claims, the indefinite articles “a” and “an” and the definite article “the” include plural as well as single referents, unless the context clearly indicates otherwise.

As used herein, the terms “comprising” and “including” can be used interchangeably. The terms “comprising” and “including” are to be interpreted as specifying the presence of the stated features or components as referred to, but does not preclude the presence or addition of one or more features, or components, or groups thereof. Additionally, the terms “comprising” and “including” are intended to include examples encompassed by the term “consisting of”. Consequently, the term “consisting of” can be used in place of the terms “comprising” and “including” to provide for more specific embodiments of the invention.

The term “consisting of” means that a subject-matter has at least 90%, 95%, 97%, 98% or 99% of the stated features or components of which it consists. In another embodiment the term “consisting of” excludes from the scope of any succeeding recitation any other features or components, excepting those that are not essential to the technical effect to be achieved.

As used herein, the term “or” is to be interpreted as an inclusive “or” meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

As used herein, and unless otherwise specified, the terms “about” and “approximately,” when used in connection with doses, amounts, or weight percents of ingredients of a composition or a dosage form, mean a dose, amount, or weight percent that is recognized by one of ordinary skill in the art to provide a pharmacological effect equivalent to that obtained from the specified dose, amount, or weight percent. In one embodiment, the terms “about” and “approximately,” when used in this context, contemplate a dose, amount, or weight percent within 30%, within 20%, within 15%, within 10%, or within 5%, of the specified dose, amount, or weight percent.

As used herein and unless otherwise specified, the terms “about” and “approximately,” when used in connection with a numeric value or a range of values which is provided to characterize a particular solid form, e.g., a specific temperature or temperature range, such as, for example, that describing a melting, dehydration, desolvation or glass transition temperature; a mass change, such as, for example, a mass change as a function of temperature or humidity; a solvent or water content, in terms of, for example, mass or a percentage; or a peak position, such as, for example, in analysis by IR or Raman spectroscopy or XRPD; indicate that the value or range of values may deviate to an extent deemed reasonable to one of ordinary skill in the art while still describing the particular solid form. For example, in particular embodiments, the terms “about” and “approximately,” when used in this context, indicate that the numeric value or range of values may vary within 25%, 20%, 15%, 10%, 9%, 8%, 7%, 6%, 5%, 4%, 3%, 2%, 1.5%, 1%, 0.5%, or 0.25% of the recited value or range of values. For example, in some embodiments, the value of XRPD peak position may vary by up to ±0.2 degrees 2θ while still describing the particular XRPD peak. In some embodiments, the value of XRPD peak position may vary by up to ±0.1 degrees 2θ. As used herein, a tilde (i.e., “˜”) preceding a numerical value or range of values indicates “about” or “approximately.”

Unless otherwise specified, the terms “X-ray powder diffraction”, “powder X-ray diffraction”, “PXRD”, and “XRPD” are used interchangeably in this application.

As used herein and unless otherwise specified, the terms “solid form” and related terms refer to a physical form which is not predominantly in a liquid or a gaseous state. As used herein, the terms “solid form” and “solid forms” encompass semi-solids. Solid forms may be crystalline, amorphous, partially crystalline, partially amorphous, or mixtures of forms.

The solid forms provided herein may have varying degrees of crystallinity or lattice order. The solid forms provided herein are not limited by any particular degree of crystallinity or lattice order, and may be 0-100% crystalline. Methods of determining the degree of crystallinity are known to those of ordinary skill in the, such as those described in Suryanarayanan, R., X-Ray Power Diffractometry, Physical Characterization of Pharmaceutical Salts, H. G. Brittain, Editor, Mercel Dekkter, Murray Hill, N.J., 1995, pp. 187-199, which is incorporated herein by reference in its entirety. In some embodiments, the solid forms provided herein are about 0, 5, 10, 15, 20, 25, 30, 35, 40, 45, 50, 55, 60, 65, 70, 75, 80, 85, 90, 95 or 100% crystalline. In some embodiments, the solid forms provided herein are at least about 0, 5, 10, 15, 20, 25, 30, 35, 40, 45, 50, 55, 60, 65, 70, 75, 80, 85, 90, 95 or 100% crystalline.

As used herein and unless otherwise specified, the term “crystalline” and related terms used herein, when used to describe a substance, component, product, or form, mean that the substance, component, product, or form is substantially crystalline, for example, as determined by X-ray diffraction. A “crystalline” state or “substantially crystalline” state is characterized by a highly ordered arrangement of the molecules, associated with which is a three-dimensional periodicity. See, e.g., Remington: The Science and Practice of Pharmacy, 21^{st} edition, Lippincott, Williams and Wilkins, Baltimore, MD (2005); The United States Pharmacopeia, 23^{rd} edition, 1843-1844 (1995).

As used herein and unless otherwise specified, the term “crystal form,” “crystal forms,” and related terms herein refer to solid forms that are crystalline. Crystal forms include single-component crystal forms and multiple-component crystal forms, and include, but are not limited to, polymorphs, solvates, hydrates, and other molecular complexes, as well as salts, solvates of salts, hydrates of salts, co-crystals of salts, other molecular complexes of salts, and polymorphs thereof. In one embodiment, a crystal form of a substance may be substantially free of amorphous forms and/or other crystal forms. In one embodiment, a crystal form of a substance may contain less than about 1%, 2%, 3%, 4%, 5%, 6%, 7%, 8%, 9%, 10%, 15%, 20%, 25%, 30%, 35%, 40%, 45% or 50% of one or more amorphous form(s) and/or other crystal form(s) on a weight basis. In one embodiment, a crystal form of a substance may be physically and/or chemically pure. In one embodiment, a crystal form of a substance may be about 99%, 98%, 97%, 96%, 95%, 94%, 93%, 92%, 91% or 90% physically and/or chemically pure.

A “single-component” solid form comprising a compound consists essentially of the compound. A “multiple-component” solid form comprising a compound comprises a significant quantity of one or more additional species, such as ions and/or molecules, within the solid form. For example, in one embodiment, a crystalline multiple-component solid form comprising a compound further comprises one or more species non-covalently bonded at regular positions in the crystal lattice. For another example, in one embodiment, an amorphous multiple-component solid form comprising a compound further comprises one or more polymer(s), and the compound is dispersed in a solid matrix that comprises the polymer(s).

Crystal forms of a substance may be obtained by a number of methods. Such methods include, but are not limited to, melt recrystallization, melt cooling, solvent recrystallization, recrystallization in confined spaces such as, e.g., in nanopores or capillaries, recrystallization on surfaces or templates such as, e.g., on polymers, recrystallization in the presence of additives, such as, e.g., co-crystal counter-molecules, desolvation, dehydration, rapid evaporation, rapid cooling, slow cooling, vapor diffusion, sublimation, grinding, and solvent-drop grinding.

Unless otherwise specified, the terms “polymorph,” “polymorphic form,” “polymorphs,” “polymorphic forms,” and related terms herein refer to two or more crystal forms that consist essentially of the same molecule, molecules or ions. Like different crystal forms, different polymorphs may have different physical properties, such as, for example, melting temperatures, heats of fusion, solubilities, dissolution rates, and/or vibrational spectra as a result of a different arrangement or conformation of the molecules or ions in the crystal lattice. The differences in physical properties exhibited by polymorphs may affect pharmaceutical parameters, such as storage stability, compressibility and density (important in formulation and product manufacturing), and dissolution rate (an important factor in bioavailability). Differences in stability can result from changes in chemical reactivity (e.g., differential oxidation, such that a dosage form discolors more rapidly when comprised of one polymorph than when comprised of another polymorph) or mechanical changes (e.g., tablets crumble on storage as a kinetically favored polymorph converts to thermodynamically a more stable polymorph) or both (e.g., tablets of one polymorph are more susceptible to breakdown at high humidity). As a result of solubility/dissolution differences, in the extreme case, some polymorphic transitions may result in lack of potency or, at the other extreme, toxicity. In addition, the physical properties of the crystal may be important in processing (for example, one polymorph might be more likely to form solvates or might be difficult to filter and wash free of impurities, and particle shape and size distribution might be different between polymorphs).

As used herein and unless otherwise specified, the term “amorphous,” “amorphous form,” and related terms used herein, mean that the substance, component or product in question is not substantially crystalline as determined by X-ray diffraction. In particular, the term “amorphous form” describes a disordered solid form, i.e., a solid form lacking long range crystalline order. In one embodiment, an amorphous form of a substance may be substantially free of other amorphous forms and/or crystal forms. In other embodiments, an amorphous form of a substance may contain less than about 1%, 2%, 3%, 4%, 5%, 10%, 15%, 20%, 25%, 30%, 35%, 40%, 45% or 50% of one or more other amorphous forms and/or crystal forms on a weight basis. In one embodiment, an amorphous form of a substance may be physically and/or chemically pure. In one embodiment, an amorphous form of a substance may be about 99%, 98%, 97%, 96%, 95%, 94%, 93%, 92%, 91% or 90% physically and/or chemically pure. In one embodiment, an amorphous form of a substance may comprise additional components or ingredients (for example, an additive, a polymer, or an excipient that may serve to further stabilize the amorphous form). In one embodiment, amorphous form may be a solid solution.

Amorphous forms of a substance can be obtained by a number of methods. Such methods include, but are not limited to, heating, melt cooling, rapid melt cooling, solvent evaporation, rapid solvent evaporation, desolvation, sublimation, grinding, ball-milling, cryo-grinding, spray drying, and freeze drying.

Unless otherwise specified, the terms “solvate” and “solvated,” as used herein, refer to a solid form of a substance which contains solvent. The terms “hydrate” and “hydrated” refer to a solvate wherein the solvent comprises water. “Polymorphs of solvates” refer to the existence of more than one solid form for a particular solvate composition. Similarly, “polymorphs of hydrates” refers to the existence of more than one solid form for a particular hydrate composition. The term “desolvated solvate,” as used herein, refers to a solid form of a substance which can be made by removing the solvent from a solvate. The terms “solvate” and “solvated,” as used herein, can also refer to a solvate of a salt, co-crystal, or molecular complex. The terms “hydrate” and “hydrated,” as used herein, can also refer to a hydrate of a salt, co-crystal, or molecular complex.

Techniques for characterizing crystal forms and amorphous forms include, but are not limited to, thermal gravimetric analysis (TGA), differential scanning calorimetry (DSC), X-ray powder diffractometry (XRPD), single-crystal X-ray diffractometry, vibrational spectroscopy, e.g., infrared (IR) and Raman spectroscopy, solid-state and solution nuclear magnetic resonance (NMR) spectroscopy, optical microscopy, hot stage optical microscopy, scanning electron microscopy (SEM), electron crystallography and quantitative analysis, particle size analysis (PSA), surface area analysis, solubility measurements, dissolution measurements, elemental analysis and Karl Fischer analysis. Characteristic unit cell parameters may be determined using one or more techniques such as, but not limited to, X-ray diffraction and neutron diffraction, including single-crystal diffraction and powder diffraction. Techniques useful for analyzing powder diffraction data include profile refinement, such as Rietveld refinement, which may be used, e.g., to analyze diffraction peaks associated with a single phase in a sample comprising more than one solid phase. Other methods useful for analyzing powder diffraction data include unit cell indexing, which allows one of skill in the art to determine unit cell parameters from a sample comprising crystalline powder. In one embodiment, an XRPD pattern is obtained using Cu Kα radiation. In one embodiment, the ramp rate (heating rate) for a DSC is about 10° C. per minute. In one embodiment, slow heating rate such as 0.5-2.0° C. per minute can be used for more accurate DSC testing. The sample pans used in a DSC testing include, e.g., aluminum, platinum, and stainless steel pans. The pans can have different configurations, e.g., open, pinhole, or hermetically-sealed pans. In one embodiment, the ramp rate for a TGA is about 10° C. per minute.

In one embodiment, the solid forms, e.g., crystal or amorphous forms, provided herein are substantially pure, i.e., substantially free of other solid forms and/or of other chemical compounds, containing less than about 25%, 20%, 15%, 10%, 9%, 8%, 7%, 6%, 5%, 4%, 3%, 2%, 1%, 0.75%, 0.5%, 0.25% or 0.1% percent by weight of one or more other solid forms and/or of other chemical compounds.

As used herein, and unless otherwise indicated, a chemical compound, solid form, or composition that is “substantially free” of another chemical compound, solid form, or composition means that the compound, solid form, or composition contains, in one embodiment, less than about 50%, 45%, 40%, 35%, 30%, 25%, 20%, 15%, 10%, 9%, 8%, 7%, 6%, 5%, 4%, 3%, 2%, 1%, 0.5%, 0.4%, 0.3%, 0.2% 0.1%, 0.05%, or 0.01% by weight of the other compound, solid form, or composition.

As used herein, and unless otherwise specified, a solid form that is “substantially physically pure” is substantially free from other solid forms. In one embodiment, a crystal form that is substantially physically pure contains less than about 50%, 45%, 40%, 35%, 30%, 25%, 20%, 15%, 10%, 9%, 8%, 7%, 6%, 5%, 4%, 3%, 2%, 1%, 0.5%, 0.4%, 0.3%, 0.2%, 0.1%, 0.05%, or 0.01% of one or more other solid forms on a weight basis. The detection of other solid forms can be accomplished by any method apparent to a person of ordinary skill in the art, including, but not limited to, diffraction analysis, thermal analysis, elemental combustion analysis and/or spectroscopic analysis.

As used herein, and unless otherwise specified, a solid form that is “substantially chemically pure” is substantially free from other chemical compounds (i.e., chemical impurities). In one embodiment, a solid form that is substantially chemically pure contains less than about 50%, 45%, 40%, 35%, 30%, 25%, 20%, 15%, 10%, 9%, 8%, 7%, 6%, 5%, 4%, 3%, 2%, 1%, 0.5%, 0.4%, 0.3%, 0.2%, 0.1%, 0.05%, or 0.01% of one or more other chemical compounds on a weight basis. The detection of other chemical compounds can be accomplished by any method apparent to a person of ordinary skill in the art, including, but not limited to, methods of chemical analysis, such as, e.g., mass spectrometry analysis, spectroscopic analysis, thermal analysis, elemental combustion analysis and/or chromatographic analysis.

Solid forms may exhibit distinct physical characterization data that are unique to a particular solid form, such as the crystal forms provided herein. These characterization data may be obtained by various techniques known to those skilled in the art, including for example X-ray powder diffraction, differential scanning calorimetry, thermal gravimetric analysis, and nuclear magnetic resonance spectroscopy. The data provided by these techniques may be used to identify a particular solid form. One skilled in the art can determine whether a solid form is one of the forms provided herein by performing one of these characterization techniques and determining whether the resulting data “matches” the reference data provided herein, which is identified as being characteristic of a particular solid form. Characterization data that “matches” those of a reference solid form is understood by those skilled in the art to correspond to the same solid form as the reference solid form. In analyzing whether data “match,” a person of ordinary skill in the art understands that particular characterization data points may vary to a reasonable extent while still describing a given solid form, due to, for example, experimental error and routine sample-to-sample analysis variation.

As used herein, and unless otherwise specified, the term “pharmaceutically acceptable salts” refers to salts prepared from pharmaceutically acceptable, relatively non-toxic acids, including inorganic acids and organic acids. In one embodiment, suitable acids include, but are not limited to, acetic, adipic, 4-aminosalicylic, ascorbic, aspartic, benzenesulfonic, benzoic, camphoric, camphorsulfonic, capric, caproic, caprylic, cinnamic, carbonic, citric, cyclamic, dihydrogenphosphoric, 2,5-dihydroxybenzoic (gentisic), 1,2-ethanedisulfonic, ethanesulfonic, fumaric, galactunoric, gluconic, glucuronic, glutamic, glutaric, glycolic, hippuric, hydrobromic, hydrochloric, hydriodic, isobutyric, isethionic, lactic, maleic, malic, malonic, mandelic, methanesulfonic, monohydrogencarbonic, monohydrogen-phosphoric, monohydrogensulfuric, mucic, 1,5-naphthalenedisulfonic, nicotinic, nitric, oxalic, pamoic, pantothenic, phosphoric, phthalic, propionic, pyroglutamic, salicylic, suberic, succinic, sulfuric, tartaric, toluenesulfonic acid, and the like (see, e.g., S. M. Berge et al., J. Pharm. Sci., 66:1-19 (1977); and Handbook of Pharmaceutical Salts: Properties, Selection and Use, P. H. Stahl and C. G. Wermuth, Eds., (2002), Wiley, Weinheim). In one embodiment, suitable acids are strong acids (e.g., with pKa less than about 1), including, but not limited to, hydrochloric, hydrobromic, sulfuric, nitric, methanesulfonic, benzene sulfonic, toluene sulfonic, naphthalene sulfonic, naphthalene disulfonic, pyridine-sulfonic, or other substituted sulfonic acids. Also included are salts of other relatively non-toxic compounds that possess acidic character, including amino acids, such as aspartic acid and the like, and other compounds, such as aspirin, ibuprofen, saccharin, and the like. Acid addition salts can be obtained by contacting the neutral form of a compound with a sufficient amount of the desired acid, either neat or in a suitable solvent. As solids, salts can exist in crystalline or amorphous forms, or mixtures thereof. Salts can also exist in polymorphic forms.

It should be noted that if there is a discrepancy between a depicted structure and a name for that structure, the depicted structure is to be accorded more weight.

As used herein and unless otherwise indicated, the term “treating” means an alleviation, in whole or in part, of a disorder, disease or condition, or one or more of the symptoms associated with a disorder, disease, or condition, or slowing or halting of further progression or worsening of those symptoms, or alleviating or eradicating the cause(s) of the disorder, disease, or condition itself.

As used herein and unless otherwise indicated, the term “preventing” means a method of delaying and/or precluding the onset, recurrence or spread, in whole or in part, of a disorder, disease or condition; barring a subject from acquiring a disorder, disease, or condition; or reducing a subject's risk of acquiring a disorder, disease, or condition.

As used herein and unless otherwise indicated, the term “managing” encompasses preventing the recurrence of the particular disease or disorder in a patient who had suffered from it, lengthening the time a patient who had suffered from the disease or disorder remains in remission, reducing mortality rates of the patients, and/or maintaining a reduction in severity or avoidance of a symptom associated with the disease or condition being managed.

As used herein and unless otherwise indicated, the term “effective amount” in connection with a compound means an amount capable of treating, preventing, or managing a disorder, disease or condition, or symptoms thereof.

As used herein and unless otherwise indicated, the term “subject” or “patient” includes an animal, including, but not limited to, an animal such a cow, monkey, horse, sheep, pig, chicken, turkey, quail, cat, dog, mouse, rat, rabbit or guinea pig, in one embodiment a mammal, in another embodiment a human.

As used herein and unless otherwise indicated, the term “relapsed” refers to a disorder, disease, or condition that responded to treatment (e.g., achieved a complete response) then had progression. The treatment can include one or more lines of therapy. In one embodiment, the disorder, disease or condition has been previously treated with one or more lines of therapy. In another embodiment, the disorder, disease or condition has been previously treated with one, two, three or four lines of therapy. In some embodiments, the disorder, disease or condition is a hematological malignancy.

As used herein and unless otherwise indicated, the term “refractory” refers to a disorder, disease, or condition that has not responded to prior treatment that can include one or more lines of therapy. In one embodiment, the disorder, disease, or condition has been previously treated one, two, three or four lines of therapy. In one embodiment, the disorder, disease, or condition has been previously treated with two or more lines of treatment, and has less than a complete response (CR) to most recent systemic therapy containing regimen. In some embodiments, the disorder, disease or condition is a hematological malignancy.

As used herein and unless otherwise indicated, the terms “co-administration” and “in combination with” include the administration of one or more therapeutic agents (for example, a compound provided herein and another anti-cancer agent or supportive care agent) either simultaneously, concurrently or sequentially with no specific time limits. In one embodiment, the agents are present in the cell or in the patient's body at the same time or exert their biological or therapeutic effect at the same time. In one embodiment, the therapeutic agents are in the same composition or unit dosage form. In another embodiment, the therapeutic agents are in separate compositions or unit dosage forms. The term “supportive care agent” refers to any substance that treats, prevents or manages an adverse effect from treatment with another therapeutic agent.

### Salts and Solid Forms Comprising Compound 1

In one embodiment, provided herein is a solid form comprising Compound 1:

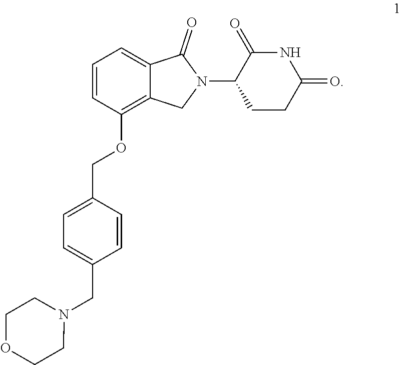 [SMILES: O=C1CC[C@H](N2Cc3c(OCc4ccc(CN5CCOCC5)cc4)cccc3C2=O)C(=O)N1]

Compound 1 has the chemical name of(S)-3-(4-((4-(morpholinomethyl)benzyl)oxy)-1-oxoisoindolin-2-yl) piperidine-2,6-dione. The synthesis and certain solid forms of Compound 1 are described in U.S. Pat. Nos. 8,518,972, 9,221,788, and 9,309,220; certain therapeutic uses of Compound 1 are described in International Patent Application Publication Nos. WO 2014/025958, WO 2014/025960, WO 2015/179276, WO 2020/072334, and WO 2021/113212; the entirety of each of which is incorporated herein by reference.

In one embodiment, the solid form comprises a free base of Compound 1. In one embodiment, the solid form comprises a salt of Compound 1. In one embodiment, the solid form comprises a hydrobromide salt of Compound 1. In one embodiment, the solid form comprises a tosylate salt of Compound 1. In one embodiment, the solid form comprises a DL-mandelate salt of Compound 1. In one embodiment, the solid form comprises a glycolate salt of Compound 1. In one embodiment, the solid form comprises a succinate salt of Compound 1. In one embodiment, the solid form comprises an L-lactate salt of Compound 1. In one embodiment, the solid form comprises a citrate salt of Compound 1.

In one embodiment, the solid form is crystalline. In one embodiment, the solid form is a hydrate. In one embodiment, the solid form is an anhydrate. In one embodiment, the solid form is a solvate. In one embodiment, the solid form is non-solvated. In one embodiment, the solid form is amorphous.

Also provided herein are salts of Compound 1. In one embodiment, the salt is a hydrobromide salt of Compound 1. In one embodiment, the salt is a tosylate salt of Compound 1. In one embodiment, the salt is a DL-mandelate salt of Compound 1. In one embodiment, the salt is a glycolate salt of Compound 1. In one embodiment, the salt is a succinate salt of Compound 1. In one embodiment, the salt is a L-lactate salt of Compound 1. In one embodiment, the salt is a citrate salt of Compound 1.

### (a) Free Base of Compound 1

In one embodiment, provided herein is a free base of Compound 1. It is contemplated that a free base of Compound 1 can exist in a variety of solid forms. Such solid forms include crystalline solids, such as polymorphs, solvates and hydrates of crystalline free base of Compound 1, as well as amorphous solids, or mixtures thereof.

In one embodiment, provided herein is a solid form comprising a free base of Compound 1. In one embodiment, the solid form is a solvate of a free base of Compound 1. In one embodiment, the solid form is a hydrate of a free base of Compound 1. In one embodiment, the solid form is an anhydrous form (anhydrate) of a free base of Compound 1.

### (i) Form A of Free Base of Compound 1

In one embodiment, provided herein is Form A of a free base of Compound 1.

In one embodiment, Form A is crystalline. In one embodiment, Form A is substantially crystalline. In one embodiment, Form A is moderately crystalline. In one embodiment, Form A is partially crystalline.

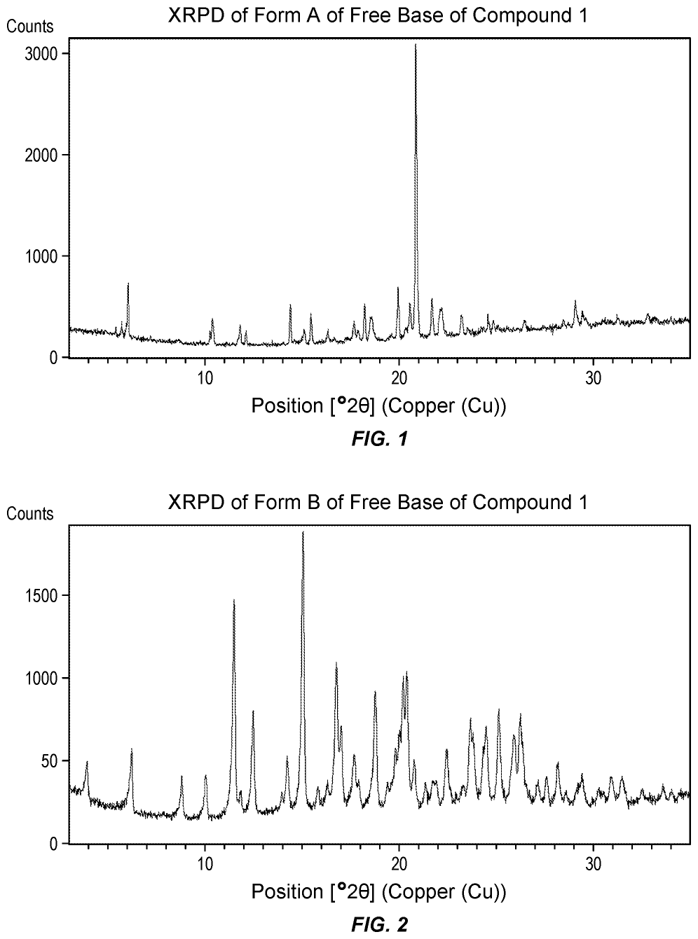

A representative XRPD pattern of Form A is provided in FIG. 1.

In one embodiment, provided herein is a solid form comprising a free base of Compound 1, characterized by 1, 2, 3, 4, 5, 6, 7, 8, 9, 10, 11, 12, 13, 14, 15, 16, 17, 18, 19, 20, 21, or all of the peaks located at approximately the following positions: 5.4, 5.7, 6.0, 10.4, 11.8, 12.1, 14.4, 15.1, 15.4, 16.3, 17.7, 18.2, 18.5, 19.9, 20.5, 20.8, 21.7, 22.2, 23.2, 24.5, 26.4, and 29.0° 20. In one embodiment, the solid form is characterized by 3 of the peaks. In one embodiment, the solid form is characterized by 5 of the peaks. In one embodiment, the solid form is characterized by 7 of the peaks. In one embodiment, the solid form is characterized by 9 of the peaks. In one embodiment, the solid form is characterized by 11 of the peaks. In one embodiment, the solid form is characterized by all of the peaks.

In one embodiment, provided herein is a solid form comprising a free base of Compound 1, characterized by an XRPD pattern comprising peaks at approximately 5.4, 5.7, and 6.0° 20. In one embodiment, the XRPD pattern further comprises peaks at approximately 10.4, 14.4, and 20.8° 20. In one embodiment, the XRPD pattern further comprises peaks at approximately 15.4, 18.2, and 19.9° 20. In one embodiment, the XRPD pattern comprises peaks at approximately 5.4, 5.7, 6.0, 10.4, 14.4, 15.4, 18.2, 19.9, 20.5, 20.8, 21.7, and 22.2° 20.

In one embodiment, provided herein is a solid form comprising a free base of Compound 1, characterized by an XRPD pattern that matches the XRPD pattern presented in FIG. 1.

In one embodiment, the XRPD patterns are obtained using Cu Kα radiation.

In one embodiment, Form A of a free base of Compound 1 is an isostructural solvate. In one embodiment, Form A of a free base of Compound 1 is a 1,4-dioxane solvate. In one embodiment, Form A of a free base of Compound 1 is a THF solvate.

In one embodiment, Form A of a free base of Compound 1 is prepared by evaporating (e.g., slow evaporating at ambient temperature) a 1,4-dioxane solution of Compound 1.

In one embodiment, Form A of a free base of Compound 1 is prepared by slurrying Compound 1 in THF. In one embodiment, the slurry is subject to a temperature cycle (e.g., between ambient temperature and about 40° C. with about 4 hour hold at each temperature).

In one embodiment, provided herein is a solid form comprising Form A of a free base of Compound 1 and amorphous free base of Compound 1. In one embodiment, provided herein is a solid form comprising Form A of a free base Compound 1 and one or more other crystalline forms of a free base of Compound 1 provided herein. In one embodiment, provided herein is a solid form comprising Form A of a free base of Compound 1 and one or more forms (e.g., amorphous or crystalline) of a salt of Compound 1 provided herein.

### (ii) Form B of Free Base of Compound 1

In one embodiment, provided herein is Form B of a free base of Compound 1.

In one embodiment, Form B is crystalline. In one embodiment, Form B is substantially crystalline. In one embodiment, Form B is moderately crystalline. In one embodiment, Form B is partially crystalline.

A representative XRPD pattern of Form B is provided in FIG. 2.

In one embodiment, provided herein is a solid form comprising a free base of Compound 1, characterized by 1, 2, 3, 4, 5, 6, 7, 8, 9, 10, 11, 12, 13, 14, 15, 16, 17, 18, 19, 20, 21, 22, 23, 24, 25, 26, or all of the peaks located at approximately the following positions: 3.9, 6.2, 8.8, 10.0, 11.5, 11.8, 12.5, 14.2, 15.0, 15.8, 16.7, 17.0, 17.7, 18.8, 19.8, 20.4, 20.8, 21.3, 22.4, 23.6, 24.4, 25.1, 25.9, 26.2, 27.1, 27.5, and 28.1° 20. In one embodiment, the solid form is characterized by 3 of the peaks. In one embodiment, the solid form is characterized by 5 of the peaks. In one embodiment, the solid form is characterized by 7 of the peaks. In one embodiment, the solid form is characterized by 9 of the peaks. In one embodiment, the solid form is characterized by 11 of the peaks. In one embodiment, the solid form is characterized by all of the peaks.

In one embodiment, provided herein is a solid form comprising a free base of Compound 1, characterized by an XRPD pattern comprising peaks at approximately 10.0, 11.5, and 12.5° 20. In one embodiment, the XRPD pattern further comprises peaks at approximately 6.2 and 15.0° 20. In one embodiment, the XRPD pattern further comprises peaks at approximately 8.8 and 16.7° 20. In one embodiment, the XRPD pattern comprises peaks at approximately 6.2, 8.8, 10.0, 11.5, 12.5, 15.0, 16.7, 18.8, 20.4, 22.4, and 25.1° 20.

In one embodiment, provided herein is a solid form comprising a free base of Compound 1, characterized by an XRPD pattern that matches the XRPD pattern presented in FIG. 2.

In one embodiment, the XRPD patterns are obtained using Cu Kα radiation.

In one embodiment, Form B of a free base of Compound 1 is an anhydrate.

In one embodiment, Form B of a free base of Compound 1 is prepared by evaporating (e.g., slow evaporating at ambient temperature) a THF solution of Compound 1.

In one embodiment, Form B of a free base of Compound 1 is prepared by slurrying Compound 1 in ethanol. In one embodiment, the slurry is subject to a temperature cycle (e.g., between ambient temperature and about 40° C. with about 4 hour hold at each temperature).

In one embodiment, provided herein is a solid form comprising Form B of a free base of Compound 1 and amorphous free base of Compound 1. In one embodiment, provided herein is a solid form comprising Form B of a free base Compound 1 and one or more other crystalline forms of a free base of Compound 1 provided herein. In one embodiment, provided herein is a solid form comprising Form B of a free base of Compound 1 and one or more forms (e.g., amorphous or crystalline) of a salt of Compound 1 provided herein.

(iii) Form C of Free Base of Compound 1

In one embodiment, provided herein is Form C of a free base of Compound 1.

In one embodiment, Form C is crystalline. In one embodiment, Form C is substantially crystalline. In one embodiment, Form C is moderately crystalline. In one embodiment, Form C is partially crystalline.

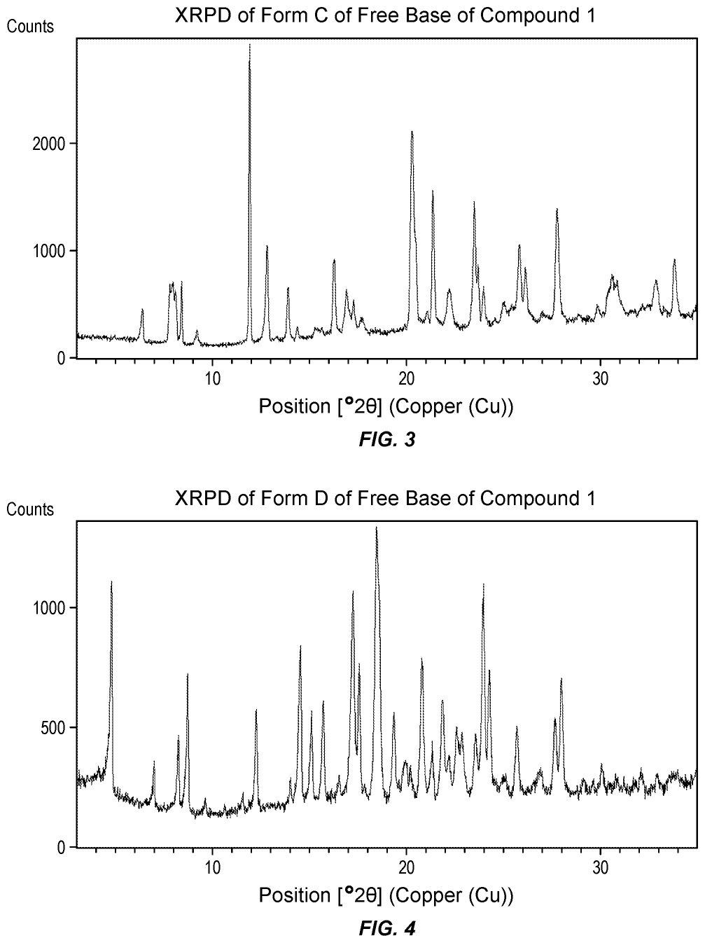

A representative XRPD pattern of Form C is provided in FIG. 3.

In one embodiment, provided herein is a solid form comprising a free base of Compound 1, characterized by 1, 2, 3, 4, 5, 6, 7, 8, 9, 10, 11, 12, 13, 14, 15, 16, 17, 18, 19, 20, 21, 22, 23, 24, 25, 26, or all of the peaks located at approximately the following positions: 6.4, 7.8, 8.1, 8.4, 9.2, 11.9, 12.8, 13.9, 14.4, 16.3, 16.9, 17.2, 17.7, 20.2, 21.0, 21.3, 22.2, 23.5, 23.9, 25.0, 25.8, 26.1, 27.7, 29.8, 30.6, 32.8, and 33.8° 20. In one embodiment, the solid form is characterized by 3 of the peaks. In one embodiment, the solid form is characterized by 5 of the peaks. In one embodiment, the solid form is characterized by 7 of the peaks. In one embodiment, the solid form is characterized by 9 of the peaks. In one embodiment, the solid form is characterized by 11 of the peaks. In one embodiment, the solid form is characterized by all of the peaks.

In one embodiment, provided herein is a solid form comprising a free base of Compound 1, characterized by an XRPD pattern comprising peaks at approximately 6.4, 7.8, and 16.3° 20. In one embodiment, the XRPD pattern further comprises peaks at approximately 8.4, 13.9, and 21.3° 20. In one embodiment, the XRPD pattern further comprises peaks at approximately 11.9, 12.8, and 20.2° 20. In one embodiment, the XRPD pattern comprises peaks at approximately 6.4, 7.8, 8.4, 11.9, 12.8, 13.9, 16.3, 20.2, 21.3, 23.5, and 27.7° 20.

In one embodiment, provided herein is a solid form comprising a free base of Compound 1, characterized by an XRPD pattern that matches the XRPD pattern presented in FIG. 3.

In one embodiment, the XRPD patterns are obtained using Cu Kα radiation.

In one embodiment, Form C of a free base of Compound 1 is a hydrate.

In one embodiment, Form C of a free base of Compound 1 is prepared by slurrying Compound 1 in a mixed solvent of methanol and water. In one embodiment, the mixed solvent is about 95:5% v/v methanol:water. In one embodiment, the slurry is subject to a temperature cycle (e.g., between ambient temperature and about 40° C. with about 4 hour hold at each temperature) for a period of time (e.g., about 3 days).

In one embodiment, provided herein is a solid form comprising Form C of a free base of Compound 1 and amorphous free base of Compound 1. In one embodiment, provided herein is a solid form comprising Form C of a free base Compound 1 and one or more other crystalline forms of a free base of Compound 1 provided herein. In one embodiment, provided herein is a solid form comprising Form C of a free base of Compound 1 and one or more forms (e.g., amorphous or crystalline) of a salt of Compound 1 provided herein.

### (iv) Form D of Free Base of Compound 1

In one embodiment, provided herein is Form D of a free base of Compound 1.

In one embodiment, Form D is crystalline. In one embodiment, Form Dis substantially crystalline. In one embodiment, Form D is moderately crystalline. In one embodiment, Form D is partially crystalline.

A representative XRPD pattern of Form D is provided in FIG. 4.

In one embodiment, provided herein is a solid form comprising a free base of Compound 1, characterized by 1, 2, 3, 4, 5, 6, 7, 8, 9, 10, 11, 12, 13, 14, 15, 16, 17, 18, 19, 20, 21, 22, 23, 24, 25, 26, or all of the peaks located at approximately the following positions: 4.8, 7.0, 8.2, 8.7, 12.2, 14.0, 14.5, 15.1, 15.7, 16.5, 17.2, 17.5, 18.4, 19.3, 19.9, 20.8, 21.3, 21.9, 22.2, 22.6, 22.9, 23.5, 23.9, 24.3, 25.6, 27.6, and 27.9° 20. In one embodiment, the solid form is characterized by 3 of the peaks. In one embodiment, the solid form is characterized by 5 of the peaks. In one embodiment, the solid form is characterized by 7 of the peaks. In one embodiment, the solid form is characterized by 9 of the peaks. In one embodiment, the solid form is characterized by 11 of the peaks. In one embodiment, the solid form is characterized by all of the peaks.

In one embodiment, provided herein is a solid form comprising a free base of Compound 1, characterized by an XRPD pattern comprising peaks at approximately 7.0, 8.2, and 17.2° 20. In one embodiment, the XRPD pattern further comprises peaks at approximately 17.5 and 18.4° 20. In one embodiment, the XRPD pattern further comprises peaks at approximately 4.8, 8.7, and 12.2° 20. In one embodiment, the XRPD pattern comprises peaks at approximately 4.8, 7.0, 8.2, 8.7, 12.2, 14.5, 15.1, 15.7, 17.2, 17.5, 18.4, 18.6, 20.8, 21.9, and 23.9° 20.

In one embodiment, provided herein is a solid form comprising a free base of Compound 1, characterized by an XRPD pattern that matches the XRPD pattern presented in FIG. 4.

In one embodiment, the XRPD patterns are obtained using Cu Kα radiation.

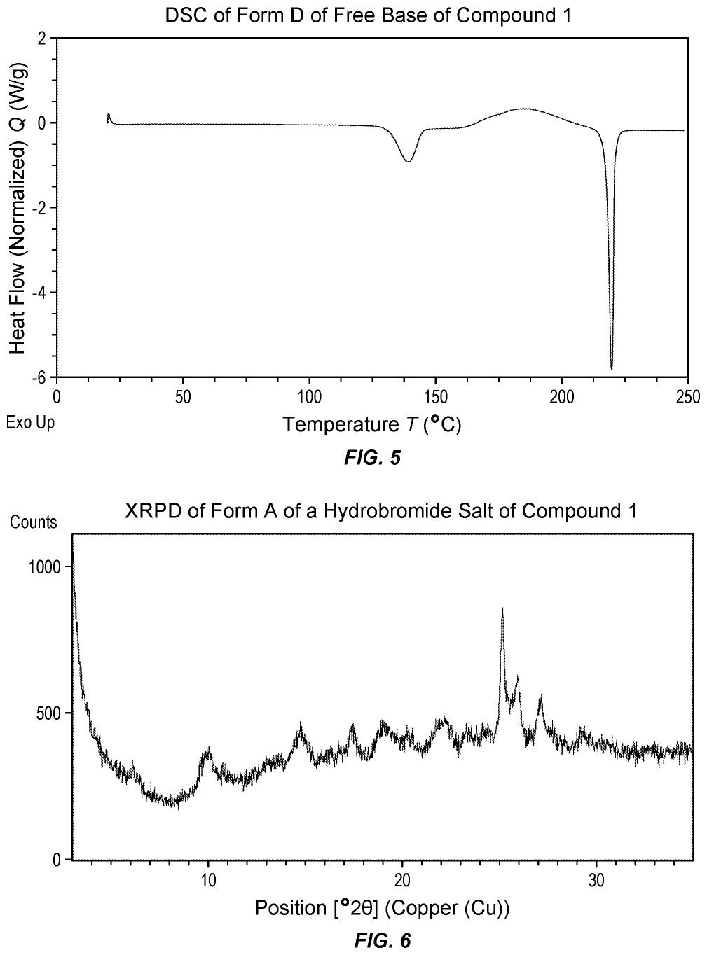

A representative differential scanning calorimetry (DSC) thermogram of Form D is provided in FIG. 5. In one embodiment, provided herein is a solid form comprising a free base of Compound 1, which exhibits, as characterized by DSC, a thermal event (endo) with a peak temperature of about 220° C. In one embodiment, the solid form also exhibits a thermal event (endo) with a peak temperature of about 139° C. In one embodiment, provided herein is a solid form comprising a free base of Compound 1, characterized by a DSC thermogram that matches the DSC thermogram presented in FIG. 5.

In one embodiment, Form D of a free base of Compound 1 is an anhydrate.

In one embodiment, Form D of a free base of Compound 1 is prepared by slurrying Compound 1 in acetonitrile. In one embodiment, the slurry is subject to a temperature cycle (e.g., between ambient temperature and about 40° C. with about 4 hour hold at each temperature) for a period of time (e.g., about 3 days).

In one embodiment, provided herein is a solid form comprising Form D of a free base of Compound 1 and amorphous free base of Compound 1. In one embodiment, provided herein is a solid form comprising Form D of a free base Compound 1 and one or more other crystalline forms of a free base of Compound 1 provided herein. In one embodiment, provided herein is a solid form comprising Form D of a free base of Compound 1 and one or more forms (e.g., amorphous or crystalline) of a salt of Compound 1 provided herein.

### (b) Hydrobromide Salt of Compound 1

In one embodiment, provided herein is a hydrobromide salt of Compound 1. It is contemplated that a hydrobromide salt of Compound 1 can exist in a variety of solid forms. Such solid forms include crystalline solids, such as polymorphs, solvates and hydrates of crystalline hydrobromide salt of Compound 1, as well as amorphous solids, or mixtures thereof.

### (i) Form A of Hydrobromide Salt of Compound 1

In one embodiment, provided herein is Form A of a hydrobromide salt of Compound 1.

In one embodiment, Form A is crystalline. In one embodiment, Form A is substantially crystalline. In one embodiment, Form A is moderately crystalline. In one embodiment, Form A is partially crystalline.

A representative XRPD pattern of Form A of a hydrobromide salt of Compound 1 is provided in FIG. 6.

In one embodiment, provided herein is a solid form comprising a hydrobromide salt of Compound 1, characterized by 1, 2, 3, 4, 5, 6, 7, 8, 9, or all of the peaks located at approximately the following positions: 6.2, 10.0, 14.8, 17.3, 19.1, 22.2, 25.2, 25.9, 27.1, and 29.2° 2θ. In one embodiment, the solid form is characterized by 3 of the peaks. In one embodiment, the solid form is characterized by 5 of the peaks. In one embodiment, the solid form is characterized by 7 of the peaks. In one embodiment, the solid form is characterized by 9 of the peaks. In one embodiment, the solid form is characterized by all of the peaks.

In one embodiment, provided herein is a solid form comprising a hydrobromide salt of Compound 1, characterized by an XRPD pattern comprising peaks at approximately 10.0, 14.8, and 17.3° 2θ. In one embodiment, the XRPD pattern further comprises peaks at approximately 19.1 and 22.2° 2θ. In one embodiment, the XRPD pattern further comprises peaks at approximately 25.2 and 25.9° 2θ. In one embodiment, the XRPD pattern comprises peaks at approximately 10.0, 14.8, 17.3, 19.1, 22.2, 25.2, 25.9, and 27.1° 2θ.

In one embodiment, provided herein is a solid form comprising a hydrobromide salt of Compound 1, characterized by an XRPD pattern that matches the XRPD pattern presented in FIG. 6.

In one embodiment, the XRPD patterns are obtained using Cu Kα radiation.

In one embodiment, Form A is a hydrate.

In one embodiment, Form A of a hydrobromide salt of Compound 1 is prepared by slurrying a hydrobromide salt of Compound 1 in THF. In one embodiment, Form A of a hydrobromide salt of Compound 1 is prepared by slurrying Compound 1 free base and hydrobromic acid in THF. In one embodiment, the slurry is subject to a temperature cycle (e.g., between ambient temperature and about 40° C. with about 4 hour hold at each temperature) for a period of time (e.g., about 3 days).

In one embodiment, provided herein is a solid form comprising Form A of a hydrobromide salt of Compound 1 and one or more forms of a free base of Compound 1 (e.g., amorphous form and crystalline forms). In one embodiment, provided herein is a solid form comprising Form A of a hydrobromide salt of Compound 1 and amorphous hydrobromide salt of Compound 1. In one embodiment, provided herein is a solid form comprising Form A of a hydrobromide salt Compound 1 and one or more other crystalline forms of a hydrobromide salt of Compound 1. In one embodiment, provided herein is a solid form comprising Form A of a hydrobromide salt of Compound 1 and one or more forms (e.g., amorphous or crystalline) of a salt of Compound 1 provided herein.

### (c) Tosylate Salt of Compound 1

In one embodiment, provided herein is a tosylate salt of Compound 1. It is contemplated that a tosylate salt of Compound 1 can exist in a variety of solid forms. Such solid forms include crystalline solids, such as polymorphs, solvates and hydrates of crystalline tosylate salt of Compound 1, as well as amorphous solids, or mixtures thereof.

### (i) Form A of Tosylate Salt of Compound 1

In one embodiment, provided herein is Form A of a tosylate salt of Compound 1.

In one embodiment, Form A is crystalline. In one embodiment, Form A is substantially crystalline. In one embodiment, Form A is moderately crystalline. In one embodiment, Form A is partially crystalline.

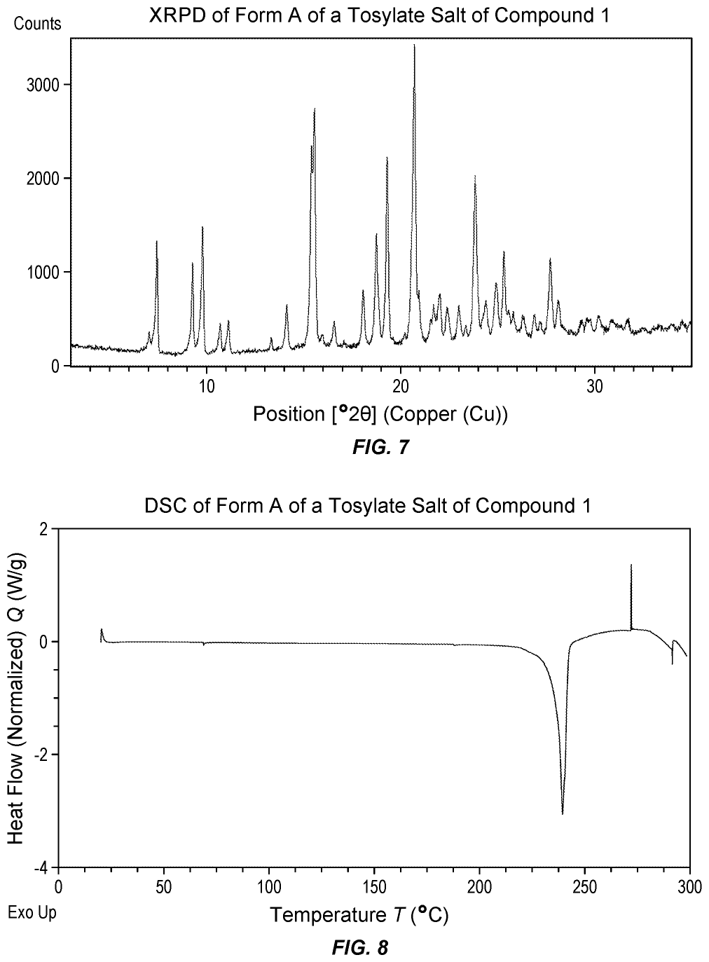

A representative XRPD pattern of Form A of a tosylate salt of Compound 1 is provided in FIG. 7.

In one embodiment, provided herein is a solid form comprising a tosylate salt of Compound 1, characterized by 1, 2, 3, 4, 5, 6, 7, 8, 9, 10, 11, 12, 13, 14, 15, 16, 17, 18, 19, 20, 21, 22, 23, 24, 25, 26, 27, 28, or all of the peaks located at approximately the following positions: 7.0, 7.4, 9.3, 9.8, 10.7, 11.1, 13.3, 14.1, 15.5, 16.6, 18.0, 18.7, 19.3, 20.7, 21.6, 22.0, 22.4, 23.0, 23.3, 23.8, 24.4, 24.9, 25.3, 25.8, 26.3, 26.9, 27.2, 27.7, and 28.1° 2θ. In one embodiment, the solid form is characterized by 3 of the peaks. In one embodiment, the solid form is characterized by 5 of the peaks. In one embodiment, the solid form is characterized by 7 of the peaks. In one embodiment, the solid form is characterized by 9 of the peaks. In one embodiment, the solid form is characterized by 11 of the peaks. In one embodiment, the solid form is characterized by all of the peaks.

In one embodiment, provided herein is a solid form comprising a tosylate salt of Compound 1, characterized by an XRPD pattern comprising peaks at approximately 9.8, 15.5, and 20.7° 2θ. In one embodiment, the XRPD pattern further comprises peaks at approximately 7.4 and 19.3° 2θ. In one embodiment, the XRPD pattern further comprises peaks at approximately 9.3 and 18.7° 2θ. In one embodiment, the XRPD pattern comprises peaks at approximately 7.4, 9.3, 9.8, 15.5, 18.7, 19.3, 20.7, 23.8, 25.3, and 27.7° 2θ.

In one embodiment, provided herein is a solid form comprising a tosylate salt of Compound 1, characterized by an XRPD pattern that matches the XRPD pattern presented in FIG. 7.

In one embodiment, the XRPD patterns are obtained using Cu Kα radiation.

A representative thermal DSC thermogram of Form A of a tosylate salt of Compound 1 is provided in FIG. 8. In one embodiment, provided herein is a solid form comprising a tosylate salt of Compound 1, which exhibits, as characterized by DSC, a thermal event (endo) with a peak temperature of about 239° C. In one embodiment, provided herein is a solid form comprising a tosylate salt of Compound 1, characterized by a DSC thermogram that matches the DSC thermogram presented in FIG. 8.

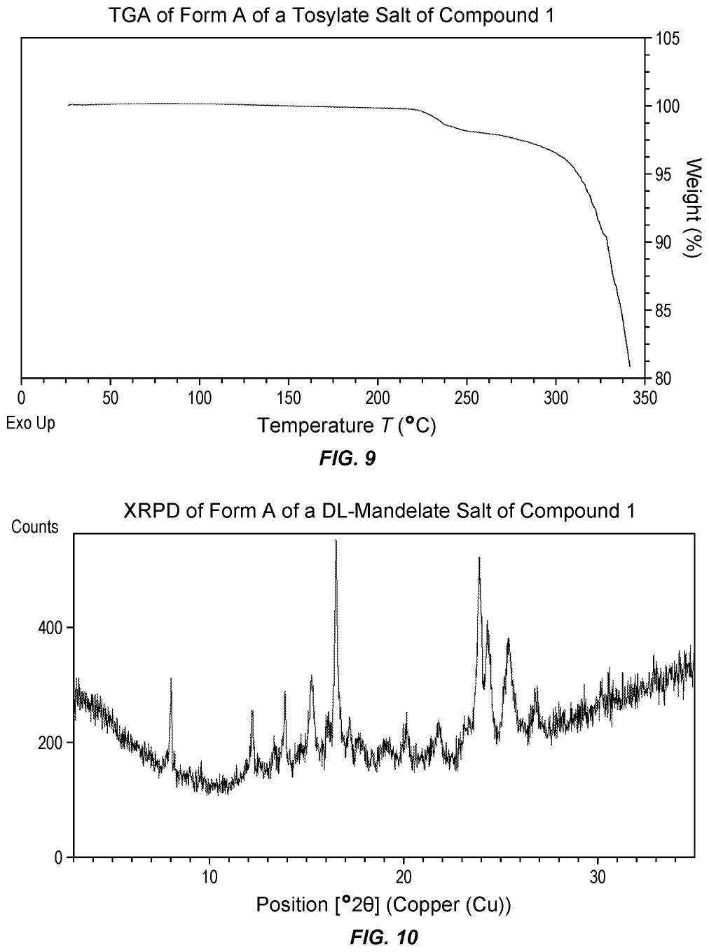

A representative thermal gravimetric analysis (TGA) thermogram of Form A of a tosylate salt of Compound 1 is provided in FIG. 9. In one embodiment, provided herein is a solid form comprising a tosylate salt of Compound 1, which exhibits a negligible weight loss upon heating from about 25° C. to about 200° C. In one embodiment, provided herein is a solid form comprising a tosylate salt of Compound 1, characterized by a TGA thermogram that matches the TGA thermogram presented in FIG. 9.

In one embodiment, the molar ratio of Compound 1 to p-toluenesulfonic acid in Form A ranges from about 2:1 to about 1:2. In one embodiment, the molar ratio of Compound 1 to p-toluenesulfonic acid in Form A is about 1:1. In one embodiment, Form A is a mono-tosylate salt of Compound 1.

In one embodiment, Form A is an anhydrate.

In one embodiment, Form A of a tosylate salt of Compound 1 is prepared by slurrying a tosylate salt of Compound 1 in ethyl acetate. In one embodiment, Form A of a tosylate salt of Compound 1 is prepared by slurrying Compound 1 free base and p-toluenesulfonic acid in ethyl acetate. In one embodiment, p-toluenesulfonic acid used can include its hydrate form, e.g., p-toluenesulfonic acid monohydrate. In one embodiment, the slurry is subject to a temperature cycle (e.g., between ambient temperature and about 40° C. with about 4 hour hold at each temperature) for a period of time (e.g., about 5 days).

In one embodiment, provided herein is a solid form comprising Form A of a tosylate salt of Compound 1 and one or more forms of a free base of Compound 1 (e.g., amorphous form and crystalline forms). In one embodiment, provided herein is a solid form comprising Form A of a tosylate salt of Compound 1 and amorphous tosylate salt of Compound 1. In one embodiment, provided herein is a solid form comprising Form A of a tosylate salt Compound 1 and one or more other crystalline forms of a tosylate salt of Compound 1. In one embodiment, provided herein is a solid form comprising Form A of a tosylate salt of Compound 1 and one or more forms (e.g., amorphous or crystalline) of a salt of Compound 1 provided herein.

### (d) DL-Mandelate Salt of Compound 1

In one embodiment, provided herein is a DL-mandelate salt of Compound 1. It is contemplated that a DL-mandelate salt of Compound I can exist in a variety of solid forms. Such solid forms include crystalline solids, such as polymorphs, solvates and hydrates of crystalline DL-mandelate salt of Compound 1, as well as amorphous solids, or mixtures thereof.

### (i) Form A of DL-Mandelate Salt of Compound 1

In one embodiment, provided herein is Form A of a DL-mandelate salt of Compound 1.

In one embodiment, Form A is crystalline. In one embodiment, Form A is substantially crystalline. In one embodiment, Form A is moderately crystalline. In one embodiment, Form A is partially crystalline.

A representative XRPD pattern of Form A of a DL-mandelate salt of Compound 1 is provided in FIG. 10.

In one embodiment, provided herein is a solid form comprising a DL-mandelate salt of Compound 1, characterized by 1, 2, 3, 4, 5, 6, 7, 8, 9, 10, 11, or all of the peaks located at approximately the following positions: 8.0, 12.2, 13.9, 15.3, 16.5, 17.2, 20.1, 21.8, 23.9, 24.3, 25.4, and 26.8° 2θ. In one embodiment, the solid form is characterized by 3 of the peaks. In one embodiment, the solid form is characterized by 5 of the peaks. In one embodiment, the solid form is characterized by 7 of the peaks. In one embodiment, the solid form is characterized by 9 of the peaks. In one embodiment, the solid form is characterized by 11 of the peaks. In one embodiment, the solid form is characterized by all of the peaks.

In one embodiment, provided herein is a solid form comprising a DL-mandelate salt of Compound 1, characterized by an XRPD pattern comprising peaks at approximately 8.0, 13.9, and 16.5° 2θ. In one embodiment, the XRPD pattern further comprises peaks at approximately 12.2 and 15.3° 2θ. In one embodiment, the XRPD pattern further comprises peaks at approximately 23.9 and 24.3° 2θ. In one embodiment, the XRPD pattern comprises peaks at approximately 8.0, 12.2, 13.9, 15.3, 16.5, 23.9, 24.3, and 25.4° 2θ.

In one embodiment, provided herein is a solid form comprising a DL-mandelate salt of Compound 1, characterized by an XRPD pattern that matches the XRPD pattern presented in FIG. 10.

In one embodiment, the XRPD patterns are obtained using Cu Kα radiation.

In one embodiment, Form A of a DL-mandelate salt of Compound 1 is prepared by slurrying a DL-mandelate salt of Compound 1 in acetone. In one embodiment, Form A of a DL-mandelate salt of Compound 1 is prepared by slurrying Compound 1 free base and DL-mandelic acid in acetone. In one embodiment, the slurry is subject to a temperature cycle (e.g., between ambient temperature and about 40° C. with about 4 hour hold at each temperature) for a period of time (e.g., about 3 days).

In one embodiment, provided herein is a solid form comprising Form A of a DL-mandelate salt of Compound 1 and one or more forms of a free base of Compound 1 (e.g., amorphous form and crystalline forms). In one embodiment, provided herein is a solid form comprising Form A of a DL-mandelate salt of Compound 1 and amorphous DL-mandelate salt of Compound 1. In one embodiment, provided herein is a solid form comprising Form A of a DL-mandelate salt Compound 1 and one or more other crystalline forms of a DL-mandelate salt of Compound 1. In one embodiment, provided herein is a solid form comprising Form A of a DL-mandelate salt of Compound 1 and one or more forms (e.g., amorphous or crystalline) of a salt of Compound 1 provided herein.

### (e) Glycolate Salt of Compound 1

In one embodiment, provided herein is a glycolate salt of Compound 1. It is contemplated that a glycolate salt of Compound 1 can exist in a variety of solid forms. Such solid forms include crystalline solids, such as polymorphs, solvates and hydrates of crystalline glycolate salt of Compound 1, as well as amorphous solids, or mixtures thereof.

### (i) Form A of Glycolate Salt of Compound 1

In one embodiment, provided herein is Form A of a glycolate salt of Compound 1.

In one embodiment, Form A is crystalline. In one embodiment, Form A is substantially crystalline. In one embodiment, Form A is moderately crystalline. In one embodiment, Form A is partially crystalline.

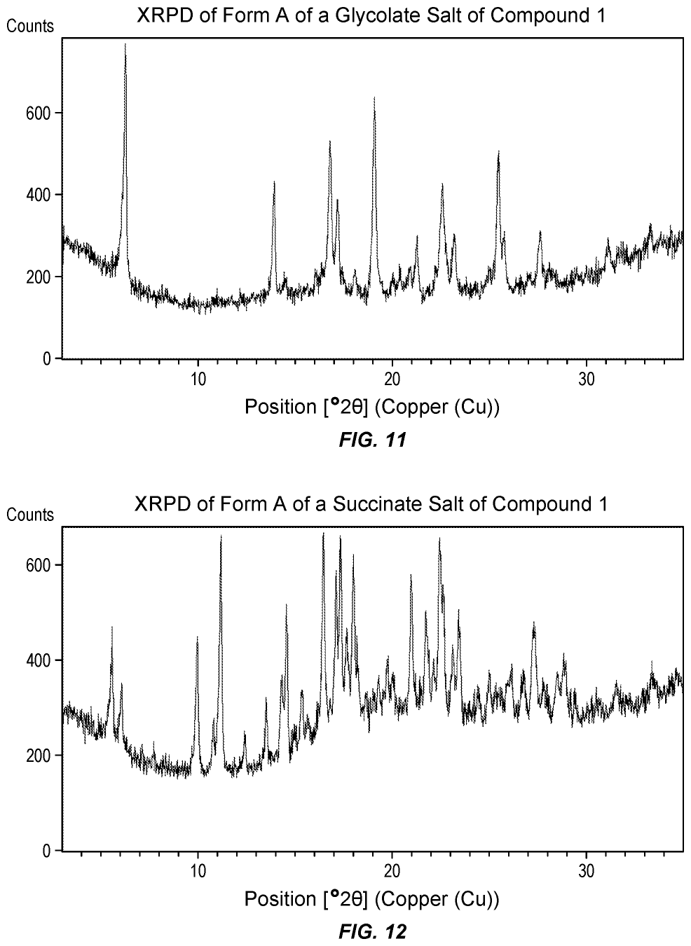

A representative XRPD pattern of Form A of a glycolate salt of Compound 1 is provided in FIG. 11.

In one embodiment, provided herein is a solid form comprising a glycolate salt of Compound 1, characterized by 1, 2, 3, 4, 5, 6, 7, 8, 9, 10, 11, or all of the peaks located at approximately the following positions: 6.3, 13.9, 16.8, 17.2, 18.1, 19.1, 21.3, 22.6, 23.2, 25.4, 27.6, and 31.1° 2θ. In one embodiment, the solid form is characterized by 3 of the peaks. In one embodiment, the solid form is characterized by 5 of the peaks. In one embodiment, the solid form is characterized by 7 of the peaks. In one embodiment, the solid form is characterized by 9 of the peaks. In one embodiment, the solid form is characterized by 11 of the peaks. In one embodiment, the solid form is characterized by all of the peaks.

In one embodiment, provided herein is a solid form comprising a glycolate salt of Compound 1, characterized by an XRPD pattern comprising peaks at approximately 6.3, 13.9, and 16.8° 2θ. In one embodiment, the XRPD pattern further comprises peaks at approximately 17.2 and 19.1° 2θ. In one embodiment, the XRPD pattern further comprises peaks at approximately 22.6 and 25.4° 2θ. In one embodiment, the XRPD pattern comprises peaks at approximately 6.3, 13.9, 16.8, 17.2, 19.1, 21.3, 22.6, 25.4, and 27.6° 2θ.

In one embodiment, provided herein is a solid form comprising a glycolate salt of Compound 1, characterized by an XRPD pattern that matches the XRPD pattern presented in FIG. 11.

In one embodiment, the XRPD patterns are obtained using Cu Kα radiation.

In one embodiment, the molar ratio of Compound 1 to glycolic acid in Form A ranges from about 2:1 to about 1:2. In one embodiment, the molar ratio of Compound 1 to glycolic acid in Form A is about 1:1. In one embodiment, Form A is a mono-glycolate salt of Compound 1.

In one embodiment, Form A is an anhydrate.

In one embodiment, Form A of a glycolate salt of Compound 1 is prepared by slurrying a glycolate salt of Compound 1 in ethyl acetate. In one embodiment, Form A of a glycolate salt of Compound 1 is prepared by slurrying Compound 1 free base and glycolic acid in ethyl acetate. In one embodiment, the slurry is subject to a temperature cycle (e.g., between ambient temperature and about 40° C. with about 4 hour hold at each temperature) for a period of time (e.g., about 3 days).

In one embodiment, provided herein is a solid form comprising Form A of a glycolate salt of Compound 1 and one or more forms of a free base of Compound 1 (e.g., amorphous form and crystalline forms). In one embodiment, provided herein is a solid form comprising Form A of a glycolate salt of Compound 1 and amorphous glycolate salt of Compound 1. In one embodiment, provided herein is a solid form comprising Form A of a glycolate salt Compound 1 and one or more other crystalline forms of a glycolate salt of Compound 1. In one embodiment, provided herein is a solid form comprising Form A of a glycolate salt of Compound 1 and one or more forms (e.g., amorphous or crystalline) of a salt of Compound 1 provided herein.

### (f) Succinate Salt of Compound 1

In one embodiment, provided herein is a succinate salt of Compound 1. It is contemplated that a succinate salt of Compound 1 can exist in a variety of solid forms. Such solid forms include crystalline solids, such as polymorphs, solvates and hydrates of crystalline succinate salt of Compound 1, as well as amorphous solids, or mixtures thereof.

### (i) Form A of Succinate Salt of Compound 1

In one embodiment, provided herein is Form A of a succinate salt of Compound 1.

In one embodiment, Form A is crystalline. In one embodiment, Form A is substantially crystalline. In one embodiment, Form A is moderately crystalline. In one embodiment, Form A is partially crystalline.

A representative XRPD pattern of Form A of a succinate salt of Compound 1 is provided in FIG. 12.

In one embodiment, provided herein is a solid form comprising a succinate salt of Compound 1, characterized by 1, 2, 3, 4, 5, 6, 7, 8, 9, 10, 11, 12, 13, 14, 15, 16, 17, 18, 19, 20, 21, or all of the peaks located at approximately the following positions: 5.6, 6.0, 9.9, 10.8, 11.2, 12.4, 13.5, 14.5, 15.3, 16.4, 17.3, 17.6, 18.0, 19.8, 20.9, 21.7, 22.4, 23.4, 26.7, 27.3, 28.4, and 28.8° 2θ. In one embodiment, the solid form is characterized by 3 of the peaks. In one embodiment, the solid form is characterized by 5 of the peaks. In one embodiment, the solid form is characterized by 7 of the peaks. In one embodiment, the solid form is characterized by 9 of the peaks. In one embodiment, the solid form is characterized by 11 of the peaks. In one embodiment, the solid form is characterized by all of the peaks.

In one embodiment, provided herein is a solid form comprising a succinate salt of Compound 1, characterized by an XRPD pattern comprising peaks at approximately 5.6, 9.9, and 11.2° 2θ. In one embodiment, the XRPD pattern further comprises peaks at approximately 14.5 and 16.4° 2θ. In one embodiment, the XRPD pattern further comprises peaks at approximately 17.6 and 18.0° 2θ. In one embodiment, the XRPD pattern comprises peaks at approximately 5.6, 9.9, 11.2, 14.5, 16.4, 17.3, 17.6, 18.0, 20.9, 22.4, and 23.4° 2θ.

In one embodiment, provided herein is a solid form comprising a succinate salt of Compound 1, characterized by an XRPD pattern that matches the XRPD pattern presented in FIG. 12.

In one embodiment, the XRPD patterns are obtained using Cu Kα radiation.

In one embodiment, the molar ratio of Compound 1 to succinic acid in Form A ranges from about 2:1 to about 1:2. In one embodiment, the molar ratio of Compound 1 to succinic acid in Form A is about 1:1. In one embodiment, Form A is a mono-succinate salt of Compound 1.

In one embodiment, Form A of a succinate salt of Compound 1 is prepared by slurrying a succinate salt of Compound 1 in ethyl acetate. In one embodiment, Form A of a succinate salt of Compound 1 is prepared by slurrying Compound 1 free base and succinic acid in ethyl acetate. In one embodiment, the slurry is subject to a temperature cycle (e.g., between ambient temperature and about 40° C. with about 4 hour hold at each temperature) for a period of time (e.g., about 3 days).

In one embodiment, provided herein is a solid form comprising Form A of a succinate salt of Compound 1 and one or more forms of a free base of Compound 1 (e.g., amorphous form and crystalline forms). In one embodiment, provided herein is a solid form comprising Form A of a succinate salt of Compound 1 and amorphous succinate salt of Compound 1. In one embodiment, provided herein is a solid form comprising Form A of a succinate salt Compound 1 and one or more other crystalline forms of a succinate salt of Compound 1. In one embodiment, provided herein is a solid form comprising Form A of a succinate salt of Compound 1 and one or more forms (e.g., amorphous or crystalline) of a salt of Compound I provided herein.

### (g) L-Lactate Salt of Compound 1

In one embodiment, provided herein is a L-lactate salt of Compound 1. It is contemplated that a L-lactate salt of Compound 1 can exist in a variety of solid forms. Such solid forms include crystalline solids, such as polymorphs, solvates and hydrates of crystalline L-lactate salt of Compound 1, as well as amorphous solids, or mixtures thereof.

### (i) Form A of L-Lactate Salt of Compound 1

In one embodiment, provided herein is Form A of a L-lactate salt of Compound 1.

In one embodiment, Form A is crystalline. In one embodiment, Form A is substantially crystalline. In one embodiment, Form A is moderately crystalline. In one embodiment, Form A is partially crystalline.

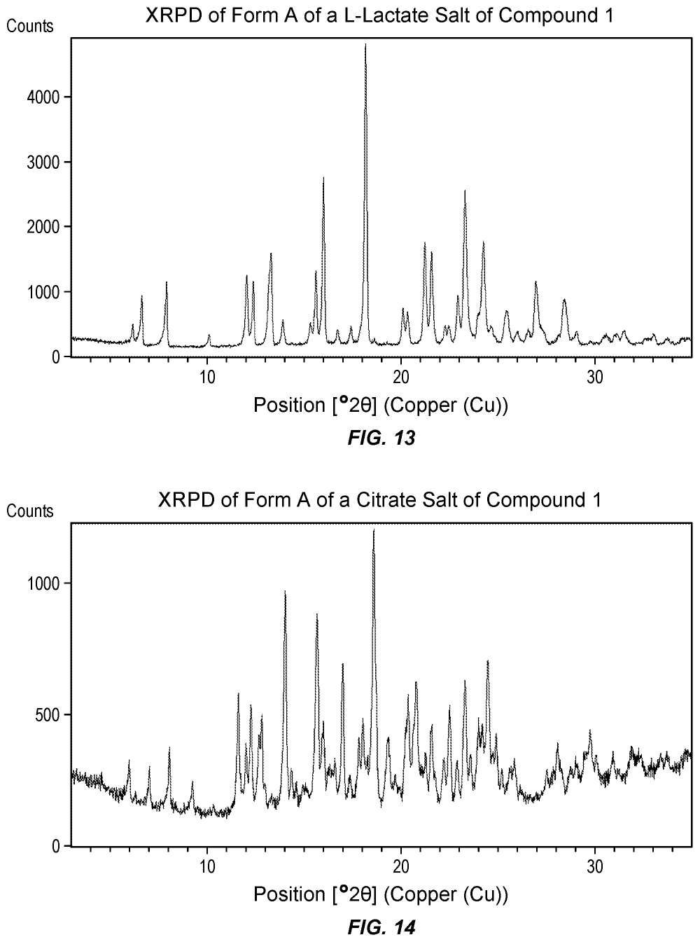

A representative XRPD pattern of Form A of a L-lactate salt of Compound 1 is provided in FIG. 13.

In one embodiment, provided herein is a solid form comprising a L-lactate salt of Compound 1, characterized by 1, 2, 3, 4, 5, 6, 7, 8, 9, 10, 11, 12, 13, 14, 15, 16, 17, 18, 19, 20, 21, 22, 23, 24, 25, or all of the peaks located at approximately the following positions: 6.2, 6.6, 7.9, 10.1, 12.0, 12.4, 13.3, 13.9, 15.3, 15.6, 16.0, 16.8, 17.4, 18.2, 20.1, 21.2, 21.6, 22.3, 22.9, 23.3, 23.9, 24.3, 25.5, 26.6, 26.9, and 28.4° 2θ. In one embodiment, the solid form is characterized by 3 of the peaks. In one embodiment, the solid form is characterized by 5 of the peaks. In one embodiment, the solid form is characterized by 7 of the peaks. In one embodiment, the solid form is characterized by 9 of the peaks. In one embodiment, the solid form is characterized by 11 of the peaks. In one embodiment, the solid form is characterized by all of the peaks.

In one embodiment, provided herein is a solid form comprising a L-lactate salt of Compound 1, characterized by an XRPD pattern comprising peaks at approximately 13.3, 16.0, and 18.2° 2θ. In one embodiment, the XRPD pattern further comprises peaks at approximately 6.6 and 7.9° 2θ. In one embodiment, the XRPD pattern further comprises peaks at approximately 12.0 and 15.6° 2θ. In one embodiment, the XRPD pattern comprises peaks at approximately 6.6, 7.9, 12.0, 12.4, 13.3, 15.6, 16.0, 18.2, 21.2, 21.6, 23.3, and 24.3° 2θ.

In one embodiment, provided herein is a solid form comprising a L-lactate salt of Compound 1, characterized by an XRPD pattern that matches the XRPD pattern presented in FIG. 13.

In one embodiment, the XRPD patterns are obtained using Cu Kα radiation.

In one embodiment, the molar ratio of Compound 1 to L-lactic acid in Form A ranges from about 2:1 to about 1:2. In one embodiment, the molar ratio of Compound 1 to L-lactic acid in Form A is about 1:1. In one embodiment, Form A is a mono-L-lactate salt of Compound 1.

In one embodiment, Form A is a hydrate.

In one embodiment, Form A of a L-lactate salt of Compound 1 is prepared by slurrying a L-lactate salt of Compound 1 in ethyl acetate. In one embodiment, Form A of a L-lactate salt of Compound 1 is prepared by slurrying Compound 1 free base and L-lactic acid in ethyl acetate. In one embodiment, the slurry is subject to a temperature cycle (e.g., between ambient temperature and about 40° C. with about 4 hour hold at each temperature) for a period of time (e.g., about 3 days).

In one embodiment, provided herein is a solid form comprising Form A of a L-lactate salt of Compound 1 and one or more forms of a free base of Compound 1 (e.g., amorphous form and crystalline forms). In one embodiment, provided herein is a solid form comprising Form A of a L-lactate salt of Compound 1 and amorphous L-lactate salt of Compound 1. In one embodiment, provided herein is a solid form comprising Form A of a L-lactate salt Compound 1 and one or more other crystalline forms of a L-lactate salt of Compound 1. In one embodiment, provided herein is a solid form comprising Form A of a L-lactate salt of Compound 1 and one or more forms (e.g., amorphous or crystalline) of a salt of Compound 1 provided herein.

### (h) Citrate Salt of Compound 1

In one embodiment, provided herein is a citrate salt of Compound 1. It is contemplated that a citrate salt of Compound 1 can exist in a variety of solid forms. Such solid forms include crystalline solids, such as polymorphs, solvates and hydrates of crystalline citrate salt of Compound 1, as well as amorphous solids, or mixtures thereof.

### (i) Form A of Citrate Salt of Compound 1

In one embodiment, provided herein is Form A of a citrate salt of Compound 1.

In one embodiment, Form A is crystalline. In one embodiment, Form A is substantially crystalline. In one embodiment, Form A is moderately crystalline. In one embodiment, Form A is partially crystalline.

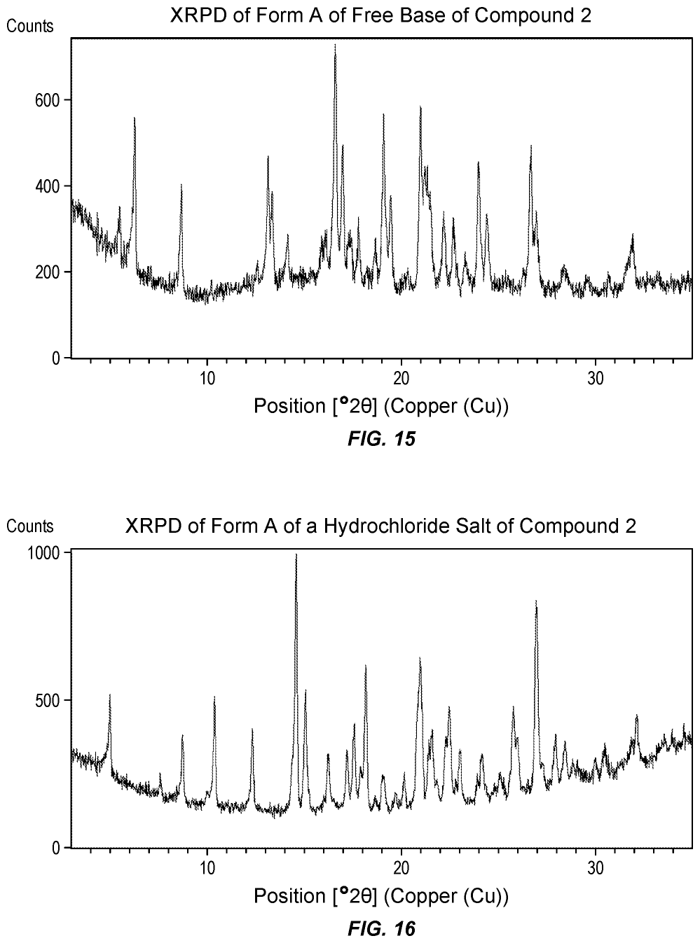

A representative XRPD pattern of Form A of a citrate salt of Compound 1 is provided in FIG. 14.

In one embodiment, provided herein is a solid form comprising a citrate salt of Compound 1, characterized by 1, 2, 3, 4, 5, 6, 7, 8, 9, 10, 11, 12, 13, 14, 15, 16, 17, 18, 19, 20, 21, 22, 23, 24, 25, 26, 27, or all of the peaks located at approximately the following positions: 6.0, 7.0, 8.1, 9.3, 11.6, 12.0, 12.3, 12.8, 14.0, 15.7, 16.0, 16.6, 17.0, 18.0, 18.6, 19.3, 20.4, 20.8, 21.6, 22.2, 22.5, 22.9, 23.3, 23.6, 24.4, 24.9, 28.1, and 29.8° 2θ. In one embodiment, the solid form is characterized by 3 of the peaks. In one embodiment, the solid form is characterized by 5 of the peaks. In one embodiment, the solid form is characterized by 7 of the peaks. In one embodiment, the solid form is characterized by 9 of the peaks. In one embodiment, the solid form is characterized by 11 of the peaks. In one embodiment, the solid form is characterized by all of the peaks.

In one embodiment, provided herein is a solid form comprising a citrate salt of Compound 1, characterized by an XRPD pattern comprising peaks at approximately 14.0, 15.7, and 18.6° 2θ. In one embodiment, the XRPD pattern further comprises peaks at approximately 11.6 and 17.0° 2θ. In one embodiment, the XRPD pattern further comprises peaks at approximately 8.1 and 12.8° 2θ. In one embodiment, the XRPD pattern comprises peaks at approximately 8.1, 11.6, 12.8, 14.0, 15.7, 17.0, 18.6, 20.8, 22.5, 23.3, and 24.4° 2θ.

In one embodiment, provided herein is a solid form comprising a citrate salt of Compound 1, characterized by an XRPD pattern that matches the XRPD pattern presented in FIG. 14.

In one embodiment, the XRPD patterns are obtained using Cu Kα radiation.

In one embodiment, the molar ratio of Compound 1 to citric acid in Form A ranges from about 2:1 to about 1:2. In one embodiment, the molar ratio of Compound 1 to citric acid in Form A is about 1:1. In one embodiment, Form A is a mono-citrate salt of Compound 1.

In one embodiment, Form A of a citrate salt of Compound 1 is prepared by slurrying a citrate salt of Compound 1 in THF. In one embodiment, Form A of a citrate salt of Compound 1 is prepared by slurrying Compound 1 free base and citric acid in THF. In one embodiment, the slurry is subject to a temperature cycle (e.g., between ambient temperature and about 40° C. with about 4 hour hold at each temperature) for a period of time (e.g., about 3 days or about 5 days).

In one embodiment, provided herein is a solid form comprising Form A of a citrate salt of Compound 1 and one or more forms of a free base of Compound 1 (e.g., amorphous form and crystalline forms). In one embodiment, provided herein is a solid form comprising Form A of a citrate salt of Compound 1 and amorphous citrate salt of Compound 1. In one embodiment, provided herein is a solid form comprising Form A of a citrate salt Compound 1 and one or more other crystalline forms of a citrate salt of Compound 1. In one embodiment, provided herein is a solid form comprising Form A of a citrate salt of Compound 1 and one or more forms (e.g., amorphous or crystalline) of a salt of Compound 1 provided herein.

### Salts and Solid Forms Comprising Compound 2

In one embodiment, provided herein is a solid form comprising Compound 2:

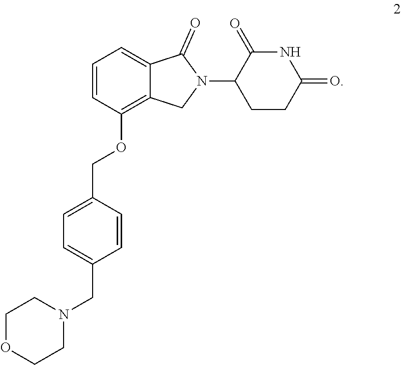 [SMILES: O=C1CCC(N2Cc3c(OCc4ccc(CN5CCOCC5)cc4)cccc3C2=O)C(=O)N1]

Compound 2 has the chemical name of 3-(4-((4-(morpholinomethyl)benzyl)oxy)-1-oxoisoindolin-2-yl) piperidine-2,6-dione. The synthesis and certain solid forms of Compound 2 are described in U.S. Pat. Nos. 8,518,972, 9,221,788, and 9,309,220; certain therapeutic uses of Compound 2 are described in International Patent Application Publication Nos. WO 2014/025958, WO 2014/025960, WO 2015/179276, WO 2020/072334, and WO 2021/113212; the entirety of each of which is incorporated herein by reference.

In one embodiment, the solid form comprises a free base of Compound 2. In one embodiment, the solid form comprises a salt of Compound 2. In one embodiment, the solid form comprises a hydrochloride salt of Compound 2.

In one embodiment, the solid form is crystalline. In one embodiment, the solid form is a hydrate. In one embodiment, the solid form is an anhydrate. In one embodiment, the solid form is a solvate. In one embodiment, the solid form is non-solvated. In one embodiment, the solid form is amorphous.

Also provided herein are salts of Compound 2. In one embodiment, the salt is a hydrochloride salt of Compound 2.

### (a) Free Base of Compound 2

In one embodiment, provided herein is a free base of Compound 2. It is contemplated that a free base of Compound 2 can exist in a variety of solid forms. Such solid forms include crystalline solids, such as polymorphs, solvates and hydrates of crystalline free base of Compound 2, as well as amorphous solids, or mixtures thereof.

In one embodiment, provided herein is a solid form comprising a free base of Compound 2. In one embodiment, the solid form is a solvate of a free base of Compound 2. In one embodiment, the solid form is a hydrate of a free base of Compound 2. In one embodiment, the solid form is an anhydrous form (anhydrate) of a free base of Compound 2.

### (i) Form A of Free Base of Compound 2

In one embodiment, provided herein is Form A of a free base of Compound 2.

In one embodiment, Form A is crystalline. In one embodiment, Form A is substantially crystalline. In one embodiment, Form A is moderately crystalline. In one embodiment, Form A is partially crystalline.

A representative XRPD pattern of Form A is provided in FIG. 15.

In one embodiment, provided herein is a solid form comprising a free base of Compound 2, characterized by 1, 2, 3, 4, 5, 6, 7, 8, 9, 10, 11, 12, 13, 14, 15, 16, or all of the peaks located at approximately the following positions: 6.3, 8.6, 13.1, 14.1, 15.8, 16.6, 16.9, 17.7, 18.6, 19.0, 19.4, 20.9, 21.2, 22.6, 23.9, 24.3, and 26.6° 2θ. In one embodiment, the solid form is characterized by 3 of the peaks. In one embodiment, the solid form is characterized by 5 of the peaks. In one embodiment, the solid form is characterized by 7 of the peaks. In one embodiment, the solid form is characterized by 9 of the peaks. In one embodiment, the solid form is characterized by 11 of the peaks. In one embodiment, the solid form is characterized by all of the peaks.

In one embodiment, provided herein is a solid form comprising a free base of Compound 2, characterized by an XRPD pattern comprising peaks at approximately 6.3, 16.6, and 19.0° 2θ. In one embodiment, the XRPD pattern further comprises peaks at approximately 8.6 and 13.1° 2θ. In one embodiment, the XRPD pattern further comprises peaks at approximately 16.9 and 20.9° 2θ. In one embodiment, the XRPD pattern comprises peaks at approximately 6.3, 8.6, 13.1, 16.6, 16.9, 19.0, 19.4, 20.9, 23.9, and 26.6° 2θ.

In one embodiment, provided herein is a solid form comprising a free base of Compound 2, characterized by an XRPD pattern that matches the XRPD pattern presented in FIG. 15.

In one embodiment, the XRPD patterns are obtained using Cu Kα radiation.

In one embodiment, Form A of a free base of Compound 2 is hydrate.

In one embodiment, Form A of a free base of Compound 2 is prepared by lyophilizing a solution of Compound 2 in a mixed solvent of 1,4-dioxane and water. In one embodiment, the mixed solvent is about 80:20 v/v 1,4-dioxane:water. In one embodiment, the lyophilization lasts from about 12 hours to about 48 hours.

In one embodiment, provided herein is a solid form comprising Form A of a free base of Compound 2 and amorphous free base of Compound 2. In one embodiment, provided herein is a solid form comprising Form A of a free base Compound 2 and one or more other crystalline forms of a free base of Compound 2. In one embodiment, provided herein is a solid form comprising Form A of a free base of Compound 2 and one or more forms (e.g., amorphous or crystalline) of a salt of Compound 2 provided herein.

### (b) Hydrochloride Salt of Compound 2

In one embodiment, provided herein is a hydrochloride salt of Compound 2. It is contemplated that a hydrochloride salt of Compound 2 can exist in a variety of solid forms. Such solid forms include crystalline solids, such as polymorphs, solvates and hydrates of crystalline hydrochloride salt of Compound 2, as well as amorphous solids, or mixtures thereof.

### (i) Form A of Hydrochloride Salt of Compound 2

In one embodiment, provided herein is Form A of a hydrochloride salt of Compound 2.

In one embodiment, Form A is crystalline. In one embodiment, Form A is substantially crystalline. In one embodiment, Form A is moderately crystalline. In one embodiment, Form A is partially crystalline.

A representative XRPD pattern of Form A of a hydrochloride salt of Compound 2 is provided in FIG. 16.

In one embodiment, provided herein is a solid form comprising a hydrochloride salt of Compound 2, characterized by 1, 2, 3, 4, 5, 6, 7, 8, 9, 10, 11, 12, 13, 14, 15, 16, 17, 18, 19, 20, 21, 22, or all of the peaks located at approximately the following positions: 5.0, 7.6, 8.8, 10.4, 12.4, 14.6, 15.1, 16.3, 17.2, 17.6, 17.9, 18.2, 19.0, 20.2, 21.0, 21.6, 22.5, 23.1, 24.2, 25.8, 27.0, 28.0, and 28.4° 2θ. In one embodiment, the solid form is characterized by 3 of the peaks. In one embodiment, the solid form is characterized by 5 of the peaks. In one embodiment, the solid form is characterized by 7 of the peaks. In one embodiment, the solid form is characterized by 9 of the peaks. In one embodiment, the solid form is characterized by all of the peaks.

In one embodiment, provided herein is a solid form comprising a hydrochloride salt of Compound 2, characterized by an XRPD pattern comprising peaks at approximately 5.0, 17.2, and 17.6° 2θ. In one embodiment, the XRPD pattern further comprises peaks at approximately 10.4 and 14.6° 2θ. In one embodiment, the XRPD pattern further comprises peaks at approximately 8.8 and 12.4° 2θ. In one embodiment, the XRPD pattern further comprises peaks at approximately 15.1 and 18.2° 2θ. In one embodiment, the XRPD pattern comprises peaks at approximately 5.0, 8.8, 10.4, 12.4, 14.6, 15.1, 17.2, 17.6, 18.2, 21.0, 22.5, 25.8, and 27.0° 2θ.

In one embodiment, provided herein is a solid form comprising a hydrochloride salt of Compound 2, characterized by an XRPD pattern that matches the XRPD pattern presented in FIG. 16.

In one embodiment, the XRPD patterns are obtained using Cu Kα radiation.

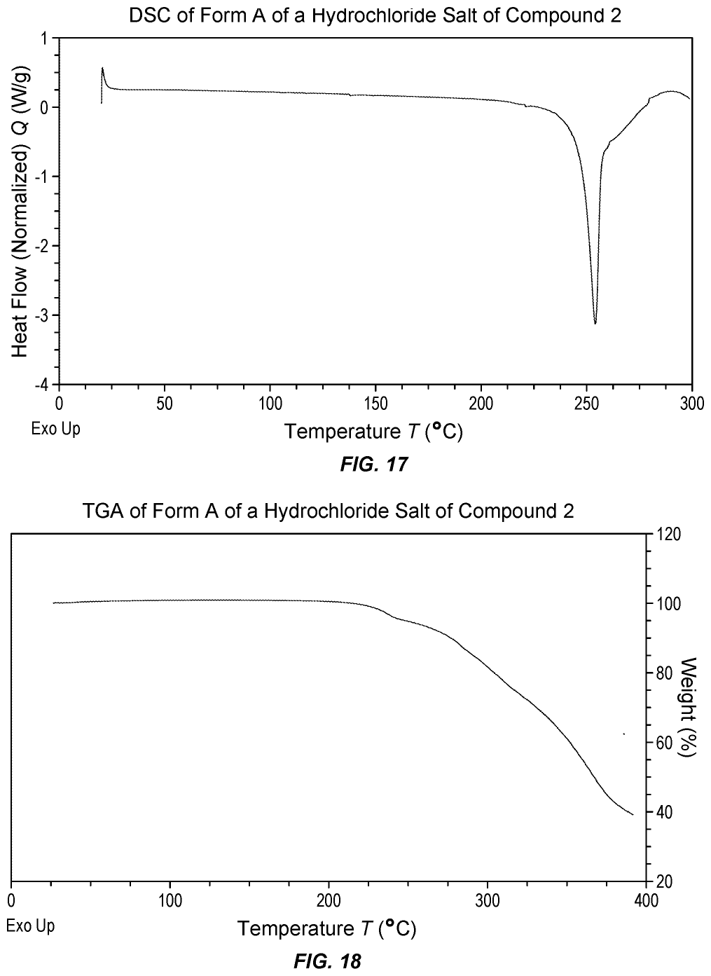

A representative thermal DSC thermogram of Form A of a hydrochloride salt of Compound 2 is provided in FIG. 17. In one embodiment, provided herein is a solid form comprising a hydrochloride salt of Compound 2, which exhibits, as characterized by DSC, a thermal event (endo) with a peak temperature of about 254° C. In one embodiment, provided herein is a solid form comprising a hydrochloride salt of Compound 2, characterized by a DSC thermogram that matches the DSC thermogram presented in FIG. 17.

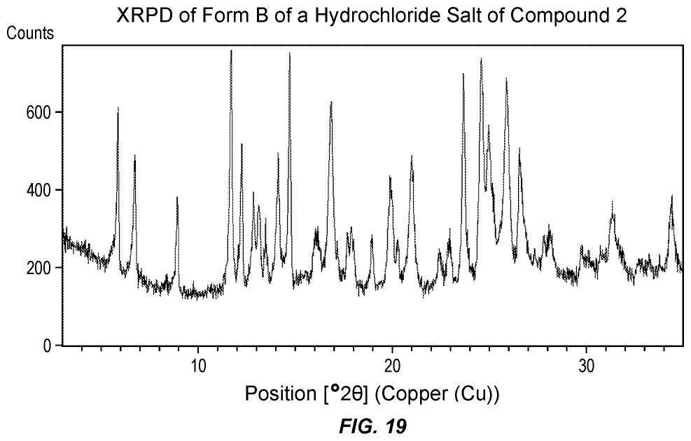

A representative TGA thermogram of Form A of a hydrochloride salt of Compound 2 is provided in FIG. 18. In one embodiment, provided herein is a solid form comprising a hydrochloride salt of Compound 2, which exhibits a negligible weight loss upon heating from about 25° C. to about 200° C. In one embodiment, provided herein is a solid form comprising a hydrochloride salt of Compound 2, characterized by a TGA thermogram that matches the TGA thermogram presented in FIG. 18.

In one embodiment, the molar ratio of Compound 2 to hydrochloric acid in Form A ranges from about 2:1 to about 1:2. In one embodiment, the molar ratio of Compound 2 to hydrochloric acid in Form A is about 1:1. In one embodiment, Form A is a mono-hydrochloride salt of Compound 2.

In one embodiment, Form A of a hydrochloride salt of Compound 2 is prepared by slurrying a hydrochloride salt of Compound 2 in acetone. In one embodiment, the slurry is subject to a temperature cycle (e.g., between ambient temperature and about 40° C. with about 4 hour hold at each temperature) for a period of time (e.g., from about 3 days to about 6 days).

In one embodiment, provided herein is a solid form comprising Form A of a hydrochloride salt of Compound 2 and one or more forms of a free base of Compound 2 (e.g., amorphous form and crystalline forms). In one embodiment, provided herein is a solid form comprising Form A of a hydrochloride salt of Compound 2 and amorphous hydrochloride salt of Compound 2. In one embodiment, provided herein is a solid form comprising Form A of a hydrochloride salt Compound 2 and one or more other crystalline forms of a hydrochloride salt of Compound 2. In one embodiment, provided herein is a solid form comprising Form A of a hydrochloride salt of Compound 2 and one or more forms (e.g., amorphous or crystalline) of a salt of Compound 2 provided herein.

### (ii) Form B of Hydrochloride Salt of Compound 2

In one embodiment, provided herein is Form B of a hydrochloride salt of Compound 2.

In one embodiment, Form B is crystalline. In one embodiment, Form B is substantially crystalline. In one embodiment, Form B is moderately crystalline. In one embodiment, Form B is partially crystalline.

A representative XRPD pattern of Form B of a hydrochloride salt of Compound 2 is provided in FIG. 19.

In one embodiment, provided herein is a solid form comprising a hydrochloride salt of Compound 2, characterized by 1, 2, 3, 4, 5, 6, 7, 8, 9, 10, 11, 12, 13, 14, 15, 16, 17, 18, 19, 20, 21, 22, 23, or all of the peaks located at approximately the following positions: 5.9, 6.7, 8.9, 11.7, 12.2, 12.8, 13.1, 13.5, 14.1, 14.7, 16.9, 18.9, 19.9, 20.3, 21.0, 22.9, 23.6, 24.6, 25.0, 25.9, 26.5, 28.1, 31.2, and 34.4, ° 2θ. In one embodiment, the solid form is characterized by 3 of the peaks. In one embodiment, the solid form is characterized by 5 of the peaks. In one embodiment, the solid form is characterized by 7 of the peaks. In one embodiment, the solid form is characterized by 9 of the peaks. In one embodiment, the solid form is characterized by all of the peaks.

In one embodiment, provided herein is a solid form comprising a hydrochloride salt of Compound 2, characterized by an XRPD pattern comprising peaks at approximately 5.9, 11.7, and 14.7° 2θ. In one embodiment, the XRPD pattern further comprises peaks at approximately 6.7 and 12.2° 2θ. In one embodiment, the XRPD pattern further comprises peaks at approximately 14.1 and 16.9° 2θ. In one embodiment, the XRPD pattern comprises peaks at approximately 5.9, 6.7, 11.7, 12.2, 14.1, 14.7, 16.9, 23.6, 24.6, and 25.9° 2θ.

In one embodiment, provided herein is a solid form comprising a hydrochloride salt of Compound 2, characterized by an XRPD pattern that matches the XRPD pattern presented in FIG. 19.

In one embodiment, the XRPD patterns are obtained using Cu Kα radiation.

In one embodiment, the molar ratio of Compound 2 to hydrochloric acid in Form B ranges from about 2:1 to about 1:2. In one embodiment, the molar ratio of Compound 2 to hydrochloric acid in Form B is about 1:1. In one embodiment, Form B is a mono-hydrochloride salt of Compound 2.

In one embodiment, Form B is a hydrate.

In one embodiment, Form B of a hydrochloride salt of Compound 2 is prepared by slurrying a hydrochloride salt of Compound 2 in water. In one embodiment, the slurry is subject to a temperature cycle (e.g., between ambient temperature and about 40° C. with about 4 hour hold at each temperature) for a period of time (e.g., from about 2 days to about 4 days).

In one embodiment, provided herein is a solid form comprising Form B of a hydrochloride salt of Compound 2 and one or more forms of a free base of Compound 2 (e.g., amorphous form and crystalline forms). In one embodiment, provided herein is a solid form comprising Form B of a hydrochloride salt of Compound 2 and amorphous hydrochloride salt of Compound 2. In one embodiment, provided herein is a solid form comprising Form B of a hydrochloride salt Compound 2 and one or more other crystalline forms of a hydrochloride salt of Compound 2. In one embodiment, provided herein is a solid form comprising Form B of a hydrochloride salt of Compound 2 and one or more forms (e.g., amorphous or crystalline) of a salt of Compound 2 provided herein.

### Methods of Use

In one embodiment, provided herein are methods of treating, preventing, and/or managing various diseases or disorders using a compound (including, e.g., a solid form or a salt) provided herein. In one embodiment, provided herein are methods of treating various diseases or disorders using a compound provided herein. In one embodiment, provided herein are methods of managing various diseases or disorders using a compound provided herein. In one embodiment, provided herein are methods of preventing various diseases or disorders using a compound provided herein. In one embodiment, provided herein are methods of treating and managing various diseases or disorders using a compound provided herein. In one embodiment, provided herein are methods of treating and preventing various diseases or disorders using a compound provided herein.

Examples of diseases or disorders include, but are not limited to, cancer, disorders associated with angiogenesis, immunodeficiency disorders, hemoglobinopathy and related disorders (e.g., anemia), TNFα related disorders, and other various diseases and disorders.

In one embodiment, provided herein is a method of treating, preventing, and/or managing an immune-related or inflammatory disease, disorder, or condition, comprising administering to a subject a therapeutically effective amount of a treatment compound provided herein. In one embodiment, provided herein is a method of treating an immune-related or inflammatory disease in a subject having the disease, comprising administering to the subject a therapeutically effective amount of a solid form provided herein.

In one embodiment, the disease is lupus, scleroderma, lupus pernio, sarcoidosis, Sjögren syndrome, ANCA-induced vasculitis, anti-phospholipid syndrome, or myasthenia gravis. In one embodiment, the disease is lupus, scleroderma, or Sjögren syndrome. In one embodiment, the disease is lupus or scleroderma. In one embodiment, the disease is systemic lupus erythematosus. In one embodiment, the disease is cutaneous lupus erythematosus. In one embodiment, the disease is scleroderma.

In one embodiment, provided herein is a method of treating, preventing, and/or managing scleroderma or a symptom thereof, comprising administering to a subject having scleroderma a therapeutically effective amount of a treatment compound provided herein. In one embodiment, provided herein is a method of treating, preventing, and/or managing scleroderma or a symptom thereof, comprising administering to a subject having scleroderma or at risk of having scleroderma a therapeutically effective amount of a treatment provided herein.

In one embodiment, the scleroderma is localized, systemic, limited, or diffuse scleroderma.

In one embodiment, the systemic scleroderma comprises CREST syndrome (Calcinosis, Raynaud's syndrome, esophageal dysfunction or dysmotility, sclerodactyly, and telangiectasia). Scleroderma is also known as systemic sclerosis or progressive systemic sclerosis.

In one embodiment, the disease is Raynaud's disease. In one embodiment, systemic sclerosis comprises scleroderma lung disease, scleroderma renal crisis, cardiac manifestations, muscular weakness (including fatigue or limited CREST), gastrointestinal dysmotility and spasm, and abnormalities in the central, peripheral and autonomic nervous system (including carpal tunnel syndrome followed by trigeminal neuralgia). It also includes general disability, including depression, and impact on quality of life.

In one embodiment, limited scleroderma is limited to the hands, the face, neck, or combinations thereof.

In one embodiment, diffuse scleroderma comprises skin tightening and also occurs above the wrists (or elbows). In one embodiment, the diffuse systemic sclerosis is sine scleroderma, comprising internal organ fibrosis, but no skin tightening; or familial progressive systemic sclerosis.

In one embodiment, scleroderma is not associated with wasting, such as disease-related wasting.

In one embodiment, provided herein is a method for the reduction, inhibition, or prevention of one or more of the following symptoms of scleroderma: (i) gradual hardening, thickening, and tightening of the skin (e.g., in extremities, such as hands, face, and feet); (ii) skin discoloration; (iii) numbness of extremities; (iv) shiny skin; (v) small white lumps under the surface of the skin that erupt into a chalky white fluid; (vi) Raynaud's esophageal dysfunction (pain, numbness, and/or color changes in the hands caused by spasm of the blood vessels upon exposure to cold or emotional stress); (vii) telangiectasia (red spots on, e.g., the hands, palms, forearms, face, and lips); (viii) pain and/or stiffness of the joints; (ix) swelling of the hands and feet; (x) itching of the skin; (xi) stiffening and curling of the fingers; (xii) ulcers (sores) on the outside of certain joints, such as knuckles and elbows; (xiii) digestive problems, such as heartburn, difficulty in swallowing, diarrhea, irritable bowel, and constipation; (xiv) fatigue and weakness; (xv) shortness of breath; (xvi) arthritis; (xvii) hair loss; (xviii) internal organ problems; (xix) digital ulcers; or (xx) digital auto-amputation, comprising administering a therapeutically effective amount of a treatment provided herein to a subject in need thereof.

Without being bound to any particular theory, it is believed that the treatment provided herein compounds provided herein enhance Th1 immune response, and suppresses Th2 immune response, which may result in anti-fibrotic effects in the skin.

In one embodiment, provided herein is a method for improving or reducing the skin thickness of a subject having scleroderma, comprising administering to the subject a therapeutically effective amount of a treatment compound provided herein. In one embodiment, the skin thickness is reduced by about 20%, about 25%, about 30%, about 40%, about 50%, about 60%, about 70% about 80%, about 90% or more.

In one embodiment, provided herein is a method for achieving one or more clinical endpoints in treating a subject with scleroderma, comprising administering to the subject a therapeutically effective amount of a treatment compound provided herein.

In one embodiment, provided herein is a method for decreasing mortality, respiratory mortality and/or respiratory hospitalization of a subject having scleroderma, comprising administering to the subject a therapeutically effective amount of a treatment compound provided herein.

In one embodiment, provided herein is a method for improving the modified Rodnan skin score of a subject having scleroderma, comprising administering to the subject a therapeutically effective amount of a treatment provided herein. In one embodiment, the improvement in modified Rodnan skin score is about 5, about 10, about 15, or about 20 points or more.

In one embodiment, provided herein is a method for improving or reducing the skin thickness of a subject having scleroderma, comprising administering to the subject a therapeutically effective amount of a treatment compound provided herein. In one embodiment, the skin thickness is reduced by about 20%, about 25%, about 30%, about 40%, about 50%, about 60%, about 70% about 80%, about 90% or more.

In one embodiment, provided herein is a method for improving or reducing skin induration of a subject having scleroderma, comprising administering to the subject a therapeutically effective amount of a treatment compound provided herein.

In one embodiment, provided herein is a method for improving the dermatology quality of life index of a subject having scleroderma, comprising administering to the subject a therapeutically effective amount of a treatment compound provided herein.

In one embodiment, provided herein is a method for improving the pulmonary function of a subject having scleroderma, comprising administering to the subject a therapeutically effective amount of a treatment compound provided herein.

In one embodiment, provided herein is a method for improving the carbon monoxide diffusing capacity of a subject having scleroderma, comprising administering to the subject a therapeutically effective amount of a treatment compound provided herein. In one embodiment, the carbon monoxide diffusing capacity of a subject is improved by an improvement in the diffusing capacity of the lung for carbon monoxide (D_{LCO}) of about 10%, about 20%, about 25%, about 30%, about 40%, about 50%, about 60%, about 70% about 80%, about 90% or more.

In one embodiment, provided herein is a method for improving the Mahler Dyspnea index of a subject having scleroderma, comprising administering to the subject a therapeutically effective amount of a treatment compound provided herein. In one embodiment, the improvement in Mahler Dyspnea index is about 4, about 5, about 6, about 7, about 8, about 9, or about 10 points or more.

In one embodiment, provided herein is a method for improving the Saint George's Respiratory Questionnaire score of a subject having scleroderma, comprising administering to the subject a therapeutically effective amount of a treatment compound provided herein. In one embodiment, the improvement in Saint George's Respiratory Questionnaire score is about 4, about 8, about 12, about 16, about 20, about 24, about 28, about 32, about 36, about 40, about 44, about 48, about 52 points or more.

In one embodiment, provided herein is a method for improving the UCLA scleroderma clinical trial consortium gastrointestinal tract score of a subject having scleroderma, comprising administering to the subject a therapeutically effective amount of a treatment compound provided herein.

In one embodiment, provided herein is a method for treating or preventing digital ulcer of a subject having scleroderma, comprising administering to the subject a therapeutically effective amount of a treatment compound provided herein.

In one embodiment, provided herein is a method for improving flow-mediated dilatation of a subject having scleroderma, comprising administering to the subject a therapeutically effective amount of a treatment compound provided herein.

In one embodiment, provided herein is a method for improving or increasing the six minute walk distance of a subject having scleroderma, comprising administering to the subject a therapeutically effective amount of a treatment compound provided herein. In one embodiment, the improvement in the six minute walk distance is about 200 meters, about 250 meters, about 300 meters, about 350 meters, about 400 meters or more.

In one embodiment, provided herein is a method of treating, preventing, and/or managing lupus erythematosus or a symptom thereof, comprising administering to a subject having lupus erythematosus a therapeutically effective amount of a treatment compound provided herein.

In one embodiment, provided herein is a method of preventing lupus erythematosus or a symptom thereof, comprising administering to a subject at risk of having lupus erythematosus a therapeutically effective amount of a treatment compound provided herein.

In one embodiment, the disease is systemic lupus erythematosus (SLE), cutaneous lupus erythematosus (CLE), or drug-induced lupus.

The phrase “systemic lupus erythematosus” is interchangeably used herein with SLE and lupus and refers to all manifestations of the disease as known in the art (including remissions and flares). In SLE, abnormal hyperactivity of B lymphocytes and massive abnormal production of immunoglobulin gamma (IgG) auto-antibodies play a key role. This pathological process results in sequestration and destruction of Ig-coated cells, fixation and cleaving of complement proteins, and release of chemotaxins, vasoactive peptides and destructive enzymes into tissues (Hahn B H. Systemic Lupus Erythematosus. In: Kasper D L, Braunwald E, Fauci A S, Hauser S L, Longo D L, Jameson, J L, editors. In: Harrison's Principles of Internal Medicine (16th edition); New York (US): McGraw-Hill; 2005. pp. 1960-1967).

Symptoms of SLE vary from person to person, and may come and go. In most patients, the symptoms include joint pain and swelling. Frequently affected joints are the fingers, hands, wrists, and knees. Some patients develop arthritis. Other common symptoms include: chest pain when taking a deep breath, fatigue, fever with no other cause, general discomfort, uneasiness, or ill feeling (malaise), hair loss, mouth sores, swollen lymph nodes, sensitivity to sunlight, skin rash—a “butterfly” rash over the cheeks and bridge of the nose affects about half of people with SLE, in some patients, the rash gets worse in sunlight, and the rash may also be widespread.

Other symptoms depend on what part of the body is affected, and may include the following:
Brain and nervous system: headaches, numbness, tingling, seizures, vision problems, personality changes,
Digestive tract: abdominal pain, nausea, and vomiting,
Heart: abnormal heart rhythms (arrhythmias),
Lung: coughing up blood and difficulty breathing, and
Skin: patchy skin color, fingers that change color when cold (Raynaud's phenomenon).

Some patients only have skin symptoms. This is called discoid lupus.

In one embodiment, the disease is moderate, severe, or very severe SLE. In one embodiment, the disease is severe SLE. The term “severe SLE” as used herein refers to an SLE condition where the patient has one or more severe or life-threatening symptoms (such as hemolytic anemia, extensive heart or lung involvement, kidney disease, or central nervous system involvement).

In one embodiment, the SLE is skin predominant SLE. In one embodiment, the patient has Cutaneous Lupus Area and Severity Index (CLASI) Activity Score≥10. In one embodiment, administration of the compound provided herein results in less than 0.3 points increase from baseline in physician global assessment (PGA). In one embodiment, administration of the compound provided herein results in a decrease of 4 or greater points from baseline in Hybrid SELENA Systemic Lupus Erythematosus Disease Activity Index (SLEDAI).

In one embodiment, provided herein is a method for achieving one or more clinical endpoints in treating a subject with SLE, comprising administering to the subject a therapeutically effective amount of a treatment compound provided herein.

In one embodiment, provided herein is a method for the overall survival, objective response rate, time to progression, progression-free survival and/or time-to-treatment failure of a subject having SLE, comprising administering to the subject a therapeutically effective amount of a treatment compound provided herein.

In certain embodiment, certain treatment compounds provided herein act as an inhibitor of primary human memory CD19+ B-cell differentiation to the plasmablast stage. Without being bound to any particular theory, it is believed that certain treatment compounds provided herein block cells at a premature stage thereby decreasing the numbers of plasmablasts that are capable of producing high levels of immunoglobulin. A functional consequence of this effect is reduced immunoglobulin G (IgG) and immunoglobulin M (IgM) production in these differentiation cultures.

In one embodiment, provided herein is a method for treating, managing, or preventing an immune-related disease, disorder, or condition, comprising administering to a subject a therapeutically effective amount of a treatment compound provided herein.

In one embodiment, provided herein is a method of treating a disease, disorder, or condition caused by, or is associated with, an inappropriate or undesirable immune response, comprising administering to a subject a therapeutically effective amount of a treatment compound provided herein.

In one embodiment, provided herein is a method of treating a disease, disorder, or condition that can be treated beneficially by immunosuppression, comprising administering to a subject a therapeutically effective amount of a treatment compound provided herein.

In one embodiment, the immune-related disease, i.e., a disease, disorder, or condition caused by, or is associated with, an inappropriate or undesirable immune response, is Sjögren syndrome, ANCA-induced vasculitis, anti-phospholipid syndrome, myasthenia gravis, Addison's disease, alopecia areata, ankylosing spondylitis, antiphospholipid antibody syndrome, antiphospholipid syndrome (primary or secondary), asthma, autoimmune gastritis, autoimmune hemolytic anemia, autoimmune hepatitis, autoimmune inner ear disease, autoimmune lymphoproliferative disease, autoimmune thrombocytopenia purpura, Balo disease, Behcet's disease, bullous pemphigoid, cardiomyopathy, celiac disease, Chagas disease, chronic inflammatory demyelinating polyneuropathy, cicatrical pemphigoid (e.g., mucous membrane pemphigoid), cold agglutinin disease, degos disease, dermatitis hepatiformis, essential mixed cryoglobulinemia, Goodpasture's syndrome, Graves' disease, Guillain-Barre syndrome, Hashimoto's thyroiditis (Hashimoto's disease; autoimmune thyroditis), idiopathic pulmonary fibrosis, idiopathic thrombocytopenia purpura, IgA nephropathy, juvenile arthritis, lichen planus, Ménière disease, mixed connective tissue disease, morephea, narcolepsy, neuromyotonia, pediatric autoimmune neuropsychiatric disorders (PANDAs), pemphigus vulgaris, pernicious anemia, polyarteritis nodosa, polychondritis, polymyalgia rheumatica, primary agammaglobulinemia, primary biliary cirrhosis, Raynaud disease (Raynaud phenomenon), Reiter's syndrome, relapsing polychondritis, rheumatic fever, Sjogren's syndrome, stiff-person syndrome (Moersch-Woltmann syndrome), Takayasu's arteritis, temporal arteritis (giant cell arteritis), uveitis, vasculitis (e.g., vasculitis not associated with lupus erythematosus), vitiligo, or Wegener's granulomatosis.

In one embodiment, provided herein is a method of treating and preventing cancer, which comprises administering to a patient a compound provided herein. In another embodiment, provided herein is method of managing cancer, which comprises administering to a patient a compound provided herein. In one embodiment, provided herein is a method of treating cancer in a subject having the cancer, comprising administering to the subject a therapeutically effective amount of a solid form provided herein.

Also provided herein are methods of treating patients who have been previously treated for cancer but are non-responsive to standard therapies, as well as those who have not previously been treated. Provided herein are methods of treating patients regardless of patient's age, although some diseases or disorders are more common in certain age groups. In one embodiment, provided herein are methods of treating patients who have undergone surgery in an attempt to treat the disease or condition at issue, as well as those who have not. Because patients with cancer have heterogeneous clinical manifestations and varying clinical outcomes, the treatment given to a patient may vary, depending on his/her prognosis. The skilled clinician will be able to readily determine without undue experimentation specific secondary agents, types of surgery, and types of non-drug based standard therapy that can be effectively used to treat an individual patient with cancer.

As used herein, the term “cancer” includes, but is not limited to, solid tumors and hematological or blood borne tumors. The term “cancer” refers to disease of skin tissues, organs, blood, and vessels, including, but not limited to, cancers of the bladder, bone, blood, brain, breast, cervix, chest, colon, endrometrium, esophagus, eye, head, kidney, liver, lymph nodes, lung, mouth, neck, ovaries, pancreas, prostate, rectum, stomach, testis, throat, and uterus. Specific cancers include, but are not limited to, advanced malignancy, amyloidosis, neuroblastoma, meningioma, hemangiopericytoma, multiple brain metastase, glioblastoma multiforms, glioblastoma, brain stem glioma, poor prognosis malignant brain tumor, malignant glioma, recurrent malignant giolma, anaplastic astrocytoma, anaplastic oligodendroglioma, neuroendocrine tumor, rectal adenocarcinoma, Dukes C & D colorectal cancer, unresectable colorectal carcinoma, metastatic hepatocellular carcinoma, Kaposi's sarcoma, karotype acute myeloblastic leukemia, Hodgkin's lymphoma, non-Hodgkin's lymphoma, cutaneous T-Cell lymphoma, cutaneous B-Cell lymphoma, diffuse large B-Cell lymphoma, low grade follicular lymphoma, malignant melanoma, malignant mesothelioma, malignant pleural effusion mesothelioma syndrome, peritoneal carcinoma, papillary serous carcinoma, gynecologic sarcoma, soft tissue sarcoma, scleroderma, cutaneous vasculitis, Langerhans cell histiocytosis, leiomyosarcoma, fibrodysplasia ossificans progressive, hormone refractory prostate cancer, resected high-risk soft tissue sarcoma, unrescectable hepatocellular carcinoma, Waldenstrom's macroglobulinemia, smoldering myeloma, indolent myeloma, fallopian tube cancer, androgen independent prostate cancer, androgen dependent stage IV non-metastatic prostate cancer, hormone-insensitive prostate cancer, chemotherapy-insensitive prostate cancer, papillary thyroid carcinoma, follicular thyroid carcinoma, medullary thyroid carcinoma, and leiomyoma.

In one embodiment, the cancer is a blood borne tumor. In one embodiment, the blood borne tumor is metastatic. In one embodiment, the blood borne tumor is drug resistant. In one embodiment, the cancer is myeloma, leukemia, or lymphoma. In one embodiment, the cancer is multiple myeloma (MM), non-Hodgkin's lymphoma (NHL), or Hodgkin's lymphoma (HL). In one embodiment, the cancer is non-Hodgkin's lymphoma. In one embodiment, the non-Hodgkin's lymphoma is diffuse large B-cell lymphoma. In one embodiment, the non-Hodgkin's lymphoma is follicular lymphoma, Burkitt's lymphoma, or mantle cell lymphoma. In one embodiment, the cancer is Hodgkin's lymphoma. In one embodiment, the cancer is multiple myeloma.

In one embodiment, the cancer is a solid tumor. In one embodiment, the cancer is breast cancer, colorectal cancer, hepatocellular carcinoma, or glioblastoma. In one embodiment, the solid tumor is hepatocellular carcinoma, prostate cancer, ovarian cancer, or glioblastoma. In one embodiment, the solid tumor is glioblastoma.

In one embodiment, the cancer is relapsed or refractory. In one embodiment, the cancer is drug-resistant. In one embodiment, the cancer is drug-resistant to lenalidomide. In one embodiment, the cancer is drug-resistant to pomalidomide. In one embodiment, the cancer is metastatic.

In one embodiment, provided herein are methods of treating, preventing, and/or managing disease in patients with impaired renal function. In one embodiment, provided herein are method of treating, preventing, and/or managing cancer in patients with impaired renal function. In one embodiment, provided herein are methods of providing appropriate dose adjustments for patients with impaired renal function due to, but not limited to, disease, aging, or other patient factors.

In one embodiment, provided herein are methods of treating, preventing, and/or managing multiple myeloma, comprising administering to a patient in need thereof a therapeutically effective amount of a compound provided herein. In one embodiment, the multiple myeloma is relapsed/refractory multiple myeloma.

In one embodiment, provided herein are methods of treating, preventing, and/or managing multiple myeloma, comprising administering to a patient in need thereof a therapeutically effective amount of a compound provided herein in combination with dexamethasone. In one embodiment, the multiple myeloma is relapsed/refractory multiple myeloma. In one embodiment, the combination therapy is administered in one or more 28-day cycles. In one embodiment, the compound is administered daily for 21 days (e.g., days 1-21) followed by 7-day rest (e.g., days 22-28). In one embodiment, the combination therapy is administered in one or more 7-day cycles. In one embodiment, the compound is administered daily for 5 days (e.g., days 1-5) followed by 2-day rest (e.g., days 6-7). In one embodiment, dexamethasone is administered once every 7 days. In one embodiment, dexamethasone is administered on days 1, 8, 15, and 22 of each 28-day cycle. In one embodiment, dexamethasone is administered at a dose of from about 10 mg to about 50 mg. In one embodiment, dexamethasone is administered at a dose of from about 20 mg to about 40 mg. In one embodiment, dexamethasone is administered at a dose of 20 mg (e.g., in subjects≥75 years old). In one embodiment, dexamethasone is administered at a dose of 40 mg (e.g., in subjects≤75 years old). In one embodiment, dexamethasone is administered orally.

In one embodiment, provided herein are methods of treating, preventing, and/or managing relapsed/refractory multiple myeloma in patients with impaired renal function or a symptom thereof, comprising administering a therapeutically effective amount of the compound provided herein to a patient having relapsed/refractory multiple myeloma with impaired renal function.

In one embodiment, provided herein are methods of preventing relapsed/refractory multiple myeloma in patients with impaired renal function or a symptom thereof, comprising administering an effective amount of a compound provided herein to a patient at risk of having relapsed/refractory multiple myeloma with impaired renal function.

Provided herein are methods of treating or managing lymphoma, particularly non-Hodgkin's lymphoma. In some embodiments, provided herein are methods for the treatment or management of non-Hodgkin's lymphoma (NHL), including but not limited to, diffuse large B-cell lymphoma (DLBCL), using prognostic factors.

In one embodiment, provided herein is a method for increasing the overall survival, objective response rate, time to progression, progression-free survival and/or time-to-treatment failure of a subject having cancer, comprising administering to the subject a therapeutically effective amount of a treatment compound provided herein.

In one embodiment, the compound provided herein is administered in an amount of about 0.1 to about 3 mg per day, 0.1 to about 2.5 mg per day, 0.1 to about 2 mg per day, 0.1 to about 1 mg per day, 0.1 to about 0.5 mg per day, or 0.1 to about 0.2 mg per day. In one embodiment, the compound provided herein is administered in an amount of about 0.15 mg per day. In one embodiment, the compound provided herein is administered in an amount of about 0.3 mg per day. In one embodiment, the compound provided herein is administered in an amount of about 0.45 mg per day. In one embodiment, the compound provided herein is administered in an amount of about 1.0 mg per day. In one embodiment, the compound provided herein is administered in an amount of about 1.3 mg per day. In one embodiment, the compound provided herein is administered in an amount of about 1.6 mg per day.

In one embodiment, the therapeutically or prophylactically effective amount is about 0.1, about 0.15, about 0.2, about 0.3, about 0.45, about 0.5, about 1, about 1.3, about 1.6, or about 2 mg per day.

In one embodiment, the recommended daily dose range of a compound provided herein for the conditions described herein lie within the range of from about 0.1 mg to about 2 mg per day, or from about 0.5 mg to about 2 mg per day, preferably given as a single once-a-day dose, or in divided doses throughout a day. In some embodiments, the dosage ranges from about 1 mg to about 2 mg per day. In other embodiments, the dosage ranges from about 0.5 to about 1 mg per day. Specific doses per day include 0.1, 0.2, 0.5, 1, or 2 mg per day.

In one embodiment, the recommended starting dosage may be about 0.1, 0.15, 0.5, 1, or 2 mg per day.

In one embodiment, the therapeutically or prophylactically effective amount is from about mg/kg/day, or from about 0.01 to about 1 mg/kg/day.

The administered dose can also be expressed in units other than mg/kg/day. For example, doses for parenteral administration can be expressed as mg/m²/day. One of ordinary skill in the art would readily know how to convert doses from mg/kg/day to mg/m²/day to given either the height or weight of a subject or both (see, www.fda.gov/cder/cancer/animalframe.htm). For example, a dose of 1 mg/kg/day for a 65 kg human is approximately equal to 38 mg/m²/day.

In one embodiment, the patient to be treated with one of the methods provided herein has not been treated with anticancer therapy prior to the administration of a compound provided herein. In one embodiment, the patient to be treated with one of the methods provided herein has been treated with anticancer therapy prior to the administration of a compound provided herein. In one embodiment, the patient to be treated with one of the methods provided herein has developed drug resistance to the anticancer therapy.

Depending on the disease to be treated and the subject's condition, a compound provided herein may be administered by oral, parenteral (e.g., intramuscular, intraperitoneal, intravenous, CIV, intracisternal injection or infusion, subcutaneous injection, or implant), inhalation, nasal, vaginal, rectal, sublingual, or topical (e.g., transdermal or local) routes of administration. A compound provided herein may be formulated, alone or together, in suitable dosage unit with pharmaceutically acceptable excipients, carriers, adjuvants and vehicles, appropriate for each route of administration.

In one embodiment, the compound provided herein is administered orally. In one embodiment, the compound provided herein is administered in a capsule or tablet. In another embodiment, the compound provided herein is administered parenterally. In yet another embodiment, the compound provided herein is administered intravenously. In one embodiment, the compound provided herein is administered for 21 days followed by seven days rest in a 28 day cycle.

The compound provided herein can be delivered as a single dose such as, e.g., a single bolus injection, or oral tablets or pills; or over time, such as, e.g., continuous infusion over time or divided bolus doses over time. The compound can be administered repeatedly if necessary, for example, until the patient experiences stable disease or regression, or until the patient experiences disease progression or unacceptable toxicity. For example, stable disease for solid tumors generally means that the perpendicular diameter of measurable lesions has not increased by 25% or more from the last measurement. Response Evaluation Criteria in Solid Tumors (RECIST) Guidelines, Journal of the National Cancer Institute 92 (3): 205-216 (2000). Stable disease or lack thereof is determined by methods known in the art such as evaluation of patient symptoms, physical examination, visualization of the tumor that has been imaged using X-ray, CAT, PET, or MRI scan and other commonly accepted evaluation modalities.

The compound provided herein can be administered once daily (QD), or divided into multiple daily doses such as twice daily (BID), three times daily (TID), and four times daily (QID). In addition, the administration can be continuous (i.e., daily for consecutive days or every day), intermittent, e.g., in cycles (i.e., including days, weeks, or months of rest without drug). As used herein, the term “daily” is intended to mean that a therapeutic compound, such as the compound provided herein is administered once or more than once each day, for example, for a period of time. The term “continuous” is intended to mean that a therapeutic compound, such as the compound provided herein is administered daily for an uninterrupted period of at least 10 days to 52 weeks. The term “intermittent” or “intermittently” as used herein is intended to mean stopping and starting at either regular or irregular intervals. For example, intermittent administration of the compound provided herein is administration for one to six days per week, administration in cycles (e.g., daily administration for two to eight consecutive weeks, then a rest period with no administration for up to one week), or administration on alternate days. The term “cycling” as used herein is intended to mean that a therapeutic compound, such as the compound provided herein is administered daily or continuously but with a rest period.

In some embodiments, the frequency of administration is in the range of about a daily dose to about a monthly dose. In one embodiment, administration is once a day, twice a day, three times a day, four times a day, once every other day, twice a week, once every week, once every two weeks, once every three weeks, or once every four weeks. In one embodiment, the compound provided herein is administered once a day. In another embodiment, the compound provided herein is administered twice a day. In yet another embodiment, the compound provided herein is administered three times a day. In still another embodiment, the compound provided herein is administered four times a day.

In one embodiment, the compound provided herein is administered once per day from one day to six months, from one week to three months, from one week to four weeks, from one week to three weeks, or from one week to two weeks. In one embodiment, the compound provided herein is administered once per day for one week, two weeks, three weeks, or four weeks. In one embodiment, the compound provided herein is administered once per day for one week. In another embodiment, the compound provided herein is administered once per day for two weeks. In yet another embodiment, the compound provided herein is administered once per day for three weeks. In still another embodiment, the compound provided herein is administered once per day for four weeks.

The methods provided herein encompass treating a patient regardless of patient's age. In some embodiments, the subject is 18 years or older. In other embodiments, the subject is more than 18, 25, 35, 40, 45, 50, 55, 60, 65, or 70 years old. In other embodiments, the subject is less than 65 years old. In other embodiments, the subject is more than 65 years old.

### Second Active Agents

A compound (including, e.g., a solid form or a salt) provided herein can be combined with one or more other pharmacologically active compounds (“second active agents” or “additional active agents”) in methods and compositions provided herein. It is believed that certain combinations work synergistically in the treatment of particular types of cancer, and certain diseases and conditions provided herein. The compounds provided herein can also work to alleviate adverse effects associated with certain second active agents, and some second active agents can be used to alleviate adverse effects associated with the compounds provided herein.

In one embodiment, the additional active agent is selected from the group consisting of an alkylating agent, an adenosine analog, a glucocorticoid, a kinase inhibitor, a SYK inhibitor, a PDE3 inhibitor, a PDE7 inhibitor, doxorubicin, chlorambucil, vincristine, bendamustine, forskolin, rituximab, or a combination thereof.

In one embodiment, the additional active agent is rituximab. In one embodiment, the additional active agent is prednisone. In one embodiment, the additional active agent is hydrocortisone. In one embodiment, the additional active agent is dexamethasone. In one embodiment, the additional active agent is a combination of dexamethasone and a proteasome inhibitor. In one embodiment, the additional active agent is a combination of dexamethasone and bortezomib. In one embodiment, the additional active agent is a combination of dexamethasone and carfilzomib. In one embodiment, the additional active agent is a combination of dexamethasone and cyclophosphamide. In one embodiment, the additional active agent is a combination of dexamethasone and salinosporamide A. In one embodiment, the additional active agent is daratumumab. In one embodiment, the additional active agent is a combination of daratumumab and dexamethasone.

In one embodiment, the additional active agent is an anti-inflammatory or immunomodulatory compound.

One or more second active ingredients or agents can be used in the methods and compositions provided herein with the compounds provided herein. Second active agents can be large molecules (e.g., proteins) or small molecules (e.g., synthetic inorganic, organometallic, or organic molecules).

Examples of large molecule active agents include, but are not limited to, hematopoietic growth factors, cytokines, and monoclonal and polyclonal antibodies. In certain embodiments, large molecule active agents are biological molecules, such as naturally occurring or artificially made proteins. Proteins that are particularly useful in this disclosure include proteins that stimulate the survival and/or proliferation of hematopoietic precursor cells and immunologically active poietic cells in vitro or in vivo. Others stimulate the division and differentiation of committed erythroid progenitors in cells in vitro or in vivo. Particular proteins include, but are not limited to: interleukins, such as IL-2 (including recombinant IL-II (“rIL2”) and canarypox IL-2), IL-10, IL-12, and IL-18; interferons, such as interferon alfa-2a, interferon alfa-2b, interferon alfa-n1, interferon alfa-n3, interferon beta-I a, and interferon gamma-I b; GM-CF and GM-CSF; GC-CSF, BCG, cancer antibodies, and EPO.

Particular proteins that can be used in the methods and compositions of the disclosure include, but are not limited to: filgrastim, which is sold in the United States under the trade name NEUPOGEN® (Amgen, Thousand Oaks, CA); sargramostim, which is sold in the United States under the trade name LEUKINE® (Immunex, Seattle, WA); and recombinant EPO, which is sold in the United States under the trade name EPGEN® (Amgen, Thousand Oaks, CA).

Inhibitors of ActRII receptors or activin-ActRII inhibitors may be used in the methods and compositions provided herein. Inhibitors of ActRII receptors include ActRIIA inhibitors and ActRIIB inhibitors. Inhibitors of ActRII receptors can be polypeptides comprising activin-binding domains of ActRII. In certain embodiments, the activin-binding domain comprising polypeptides are linked to an Fc portion of an antibody (i.e., a conjugate comprising an activin-binding domain comprising polypeptide of an ActRII receptor and an Fc portion of an antibody is generated). In certain embodiments, the activin-binding domain is linked to an Fc portion of an antibody via a linker, e.g., a peptide linker. Examples of such non-antibody proteins selected for activin or ActRIIA binding and methods for design and selection of the same are found in WO/2002/088171, WO/2006/055689, WO/2002/032925, WO/2005/037989, US 2003/0133939, and US 2005/0238646, each of which is incorporated herein by reference in its entirety. In one embodiment, the inhibitor of ActRII receptors is ACE-11. In another embodiment, the inhibitor of ActRII receptors is ACE-536.

Recombinant and mutated forms of GM-CSF can be prepared as described in U.S. Pat. Nos. 5,391,485; 5,393,870; and 5,229,496; the disclosure of each of which is incorporated herein by reference in its entirety. Recombinant and mutated forms of G-CSF can be prepared as described in U.S. Pat. Nos. 4,810,643; 4,999,291; 5,528,823; and 5,580,755; the disclosure of each of which is incorporated herein by reference in its entirety.

This disclosure encompasses the use of native, naturally occurring, and recombinant proteins. The disclosure further encompasses mutants and derivatives (e.g., modified forms) of naturally occurring proteins that exhibit, in vivo, at least some of the pharmacological activity of the proteins upon which they are based. Examples of mutants include, but are not limited to, proteins that have one or more amino acid residues that differ from the corresponding residues in the naturally occurring forms of the proteins. Also encompassed by the term “mutants” are proteins that lack carbohydrate moieties normally present in their naturally occurring forms (e.g., nonglycosylated forms). Examples of derivatives include, but are not limited to, pegylated derivatives and fusion proteins, such as proteins formed by fusing IgG1 or IgG3 to the protein or active portion of the protein of interest. See, e.g., Penichet, M. L. and Morrison, S. L., J. Immunol. Methods 248:91-101 (2001).

Antibodies that can be used in combination with the compounds provided herein include monoclonal and polyclonal antibodies. Examples of antibodies include, but are not limited to, trastuzumab (HERCEPTIN®), rituximab (RITUXAN®), bevacizumab (AVASTIN™), pertuzumab (OMNITARG™), tositumomab (BEXXAR®), edrecolomab (PANOREX®), panitumumab and G250.

Large molecule active agents may be administered in the form of anti-cancer vaccines. For example, vaccines that secrete, or cause the secretion of, cytokines such as IL-2, SCF, CXCl4 (platelet factor 4), G-CSF, and GM-CSF can be used in the methods, pharmaceutical compositions, and kits provided herein. See, e.g., Emens, L. A., et al., Curr. Opinion Mol. Ther. 3 (1): 77-84 (2001).

Second active agents that are small molecules can also be used to alleviate adverse effects associated with the administration of the compounds provided herein. However, like some large molecules, many are believed to be capable of providing a synergistic effect when administered with (e.g., before, after or simultaneously) the compounds provided herein. Examples of small molecule second active agents include, but are not limited to, anti-cancer agents, antibiotics, immunosuppressive agents, and steroids.

Examples of anti-cancer agents include, but are not limited to: abraxane; ace-11; acivicin; aclarubicin; acodazole hydrochloride; acronine; adozelesin; aldesleukin; altretamine; ambomycin; ametantrone acetate; amrubicin; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; celecoxib (COX-2 inhibitor); chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine: fosquidone; fostriecin sodium; gemcitabine; gemcitabine hydrochloride: herceptin; hydroxyurea; idarubicin hydrochloride; ifosfamide; ilmofosine; iproplatin; irinotecan; irinotecan hydrochloride; lanreotide acetate; lapatinib; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; romidepsin; safingol; safingol hydrochloride; semustine; simtrazene; sparfosate sodium; sparsomycin; spirogermanium hydrochloride; spiromustine; spiroplatin; stem cell treatments such as PDA-001; streptonigrin; streptozocin; sulofenur; talisomycin; tecogalan sodium; taxotere; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; and zorubicin hydrochloride.

Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABI, antagonists; benzochlorins; benzoylstaurosporine; beta lactam derivatives; beta-alethine; betaclamycin B; betulinic acid; b-FGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; doxorubicin; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imatinib (e.g., GLEEVEC®), imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mitoguazone; mitolactol; mitomycin analogues; mitonafide; mitotoxin fibroblast growth factor-saporin; mitoxantrone; mofarotene; molgramostim; Erbitux, human chorionic gonadotrophin; monophosphoryl lipid A+myobacterium cell wall sk; mopidamol; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; nilutamide; nisamycin; nitric oxide modulators; nitroxide antioxidant; nitrullyn; oblimersen (GENASENSE®); O⁶-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stipiamide; stromelysin inhibitors; sulfinosine; superactive vasoactive intestinal peptide antagonist; suradista; suramin; swainsonine; tallimustine; tamoxifen methiodide; tauromustine; tazarotene; tecogalan sodium; tegafur; tellurapyrylium; telomerase inhibitors; temoporfin; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; velaresol; veramine; verdins; verteporfin; vinorelbine; vinxaltine; vitaxin; vorozole; zanoterone; zeniplatin; zilascorb; and zinostatin stimalamer.

In one embodiment, the second active agent is proteasome inhibitor. In one embodiment, the proteasome inhibitor is bortezomib, disulfiram, epigallocatechin-3-gallate, salinosporamide A, carfilzomib, ONX 0912, CEP-18770, or MLN9708.

In one embodiment, the second active agent is HDAC inhibitor. In one embodiment, the HDAC inhibitor is vorinostat, romidepsin, panobinostat, valproic acid, belinostat, mocetinostat, abexinostat, entinostat, SB939, resminostat, givinostat, CUDC-101, AR-42, CHR-2845, CHR-3996, 4SC-202, CG200745, ACY-1215, sulforaphane, kevetrin, or trichostatin A.

In one embodiment, the second active agent is mitotic inhibitor. In one embodiment, the mitotic inhibitor is taxanes, vinca alkaloids, or colchicines. In one embodiment, the taxane is paclitaxel (Abraxane) or docetaxel. In one embodiment, the vinca alkaloid is vinblastine, vincristine, vindesine, or vinorelbine.

Specific second active agents include, but are not limited to, oblimersen (GENASENSE®), remicade, docetaxel, celecoxib, melphalan, dexamethasone (DECADRON®), steroids, gemcitabine, cisplatinum, temozolomide, etoposide, cyclophosphamide, temodar, carboplatin, procarbazine, gliadel, tamoxifen, topotecan, methotrexate, ARISA®, taxol, taxotere, fluorouracil, leucovorin, irinotecan, xeloda, CPT-11, interferon alpha, pegylated interferon alpha (e.g., PEG INTRON-A), capecitabine, cisplatin, thiotepa, fludarabine, carboplatin, liposomal daunorubicin, cytarabine, doxetaxol, pacilitaxel, vinblastine, IL-2, GM-CSF, dacarbazine, vinorelbine, zoledronic acid, palmitronate, biaxin, busulphan, prednisone, bisphosphonate, arsenic trioxide, vincristine, doxorubicin (DOXIL®), paclitaxel, ganciclovir, adriamycin, estramustine sodium phosphate (EMCYT®), sulindac, and etoposide.

Use with Transplantation Therapy

The compound (including, e.g., a solid form or a salt) provided herein can be used to reduce the risk of Graft Versus Host Disease (GVHD). Therefore, encompassed herein is a method of treating, preventing and/or managing cancer, which comprises administering the compound provided herein in conjunction with transplantation therapy.

As those of ordinary skill in the art are aware, the treatment of cancer is often based on the stages and mechanism of the disease. For example, as inevitable leukemic transformation develops in certain stages of cancer, transplantation of peripheral blood stem cells, hematopoietic stem cell preparation or bone marrow may be necessary. The combined use of the compound provided herein and transplantation therapy provides a unique and unexpected synergism. In particular, the compound provided herein exhibits immunomodulatory activity that may provide additive or synergistic effects when given concurrently with transplantation therapy in patients with cancer.

The compound provided herein can work in combination with transplantation therapy reducing complications associated with the invasive procedure of transplantation and risk of GVHD. Encompassed herein is a method of treating, preventing and/or managing cancer which comprises administering to a patient (e.g., a human) the compound provided herein before, during, or after the transplantation of umbilical cord blood, placental blood, peripheral blood stem cell, hematopoietic stem cell preparation, or bone marrow. Some examples of stem cells suitable for use in the methods provided herein are disclosed in U.S. Pat. No. 7,498,171, the disclosure of which is incorporated herein by reference in its entirety.

In one embodiment, the compound provided herein is administered to patients with multiple myeloma before, during, or after the transplantation of autologous peripheral blood progenitor cell.

In another embodiment, the compound provided herein is administered to patients with relapsing multiple myeloma after the stem cell transplantation.

In yet another embodiment, the compound provided herein and prednisone are administered as maintenance therapy to patients with multiple myeloma following the transplantation of autologous stem cell.

In yet another embodiment, the compound provided herein and dexamethasone are administered as salvage therapy for low risk post transplantation to patients with multiple myeloma.

In yet another embodiment, the compound provided herein and dexamethasone are administered as maintenance therapy to patients with multiple myeloma following the transplantation of autologous bone marrow.

In yet another embodiment, the compound provided herein is administered following the administration of high dose of melphalan and the transplantation of autologous stem cell to patients with chemotherapy responsive multiple myeloma.

In yet another embodiment, the compound provided herein and PEG INTRO-A are administered as maintenance therapy to patients with multiple myeloma following the transplantation of autologous CD34-selected peripheral stem cell.

In yet another embodiment, the compound provided herein is administered with post transplant consolidation chemotherapy to patients with newly diagnosed multiple myeloma to evaluate anti-angiogenesis.

In still another embodiment, the compound provided herein and dexamethasone are administered as maintenance therapy after DCEP consolidation, following the treatment with high dose of melphalan and the transplantation of peripheral blood stem cell to 65 years of age or older patients with multiple myeloma.

In one embodiment, the compound provided herein is administered to patients with NHL (e.g., DLBCL) before, during, or after the transplantation of autologous peripheral blood progenitor cell.

In another embodiment, the compound provided herein is administered to patients with NHL (e.g., DLBCL) after a stem cell transplantation.

### Cycling Therapy

In certain embodiments, the prophylactic or therapeutic agents provided herein are cyclically administered to a patient. Cycling therapy involves the administration of an active agent for a period of time, followed by a rest for a period of time, and repeating this sequential administration. Cycling therapy can reduce the development of resistance to one or more of the therapies, avoid, or reduce the side effects of one of the therapies, and/or improves the efficacy of the treatment.

Consequently, in certain embodiments, the compound (including, e.g., a solid form or a salt) provided herein is administered daily in a single or divided doses in a four to six week cycle with a rest period of about a week or two weeks. The cycling method further allows the frequency, number, and length of dosing cycles to be increased. Thus, encompassed herein in certain embodiments is the administration of a compound provided herein for more cycles than are typical when it is administered alone. In certain embodiments, a compound provided herein is administered for a greater number of cycles that would typically cause dose-limiting toxicity in a patient to whom a second active ingredient is not also being administered.

In one embodiment, the compound is administered daily and continuously for three or four weeks at a dose of from about 0.1 to about 150 mg/d followed by a break of one or two weeks.

In another embodiment, the compound provided herein and a second active ingredient are administered orally, with administration of the compound occurring 30 to 60 minutes prior to a second active ingredient, during a cycle of four to six weeks. In certain embodiments, the combination of the compound provided herein and a second active ingredient is administered by intravenous infusion over about 90 minutes every cycle. In certain embodiments, one cycle comprises the administration from about 0.1 to about 150 mg/day of the compound provided herein and from about 50 to about 200 mg/m²/day of a second active ingredient daily for three to four weeks and then one or two weeks of rest. In certain embodiments, the number of cycles during which the combinatorial treatment is administered to a patient is ranging from about one to about 24 cycles, from about two to about 16 cycles, or from about four to about three cycles.

### Pharmaceutical Compositions and Routes of Administration

In one embodiment, provided herein are pharmaceutical compositions and dosage forms, which comprise a solid form provided herein. In another embodiment, pharmaceutical compositions and dosage forms further comprise one or more excipients. In certain embodiment, pharmaceutical compositions and dosage forms provided herein also comprise one or more additional active agents in amounts effective for achieving a modulation of disease or disease symptoms, including those described herein.

In one embodiment, the pharmaceutical compositions provided herein may be administered orally, parenterally, by inhalation spray, topically, rectally, nasally, buccally, vaginally or via an implanted reservoir, preferably by oral administration or administration by injection. Oral delivery formats include, but are not limited to, tablets, capsules, caplets, solutions, suspensions, and syrups, and may also comprise a plurality of granules, beads, powders or pellets that may or may not be encapsulated. In one embodiment, the pharmaceutical compositions may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intraarterial, intrasynovial, intrasternal, intrathecal, intralesional and intracranial injection or infusion techniques.

In one embodiment, dosage forms provided herein comprising a solid form provided herein are suitable for oral, mucosal (e.g., nasal, sublingual, vaginal, buccal, or rectal), parenteral (e.g., subcutaneous, intravenous, bolus injection, intramuscular, or intraarterial), topical (e.g., eye drops or other ophthalmic preparations), transdermal, or transcutaneous administration to a patient. Examples of dosage forms include, but are not limited to: tablets; caplets; capsules, such as soft elastic gelatin capsules; cachets; troches; lozenges; dispersions; suppositories; powders; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; eye drops or other ophthalmic preparations suitable for topical administration; and sterile solids (e.g., crystalline or amorphous solids) that can be reconstituted to provide liquid dosage forms suitable for parenteral administration to a patient.

Whether a particular excipient is suitable for incorporation into a pharmaceutical composition or dosage form provided herein depends on a variety of factors, including, but not limited to, the route of administration. For example, oral dosage forms such as tablets may contain excipients not suited for use in parenteral dosage forms. The suitability of a particular excipient may also depend on the specific active ingredients in the dosage form. For example, the decomposition of some active ingredients may be accelerated by some excipients such as lactose, or when exposed to water. Active ingredients that comprise primary or secondary amines are particularly susceptible to such accelerated decomposition. Consequently, encompassed herein are pharmaceutical compositions and dosage forms that contain little, if any, lactose. As used herein, the term “lactose-free” means that the amount of lactose present, if any, is insufficient to substantially increase the degradation rate of an active ingredient.

Lactose-free compositions provided herein can comprise excipients that are listed, for example, in the U.S. Pharmacopeia (USP) 25-NF20 (2002). In one embodiment, lactose-free compositions comprise active ingredients, a binder/filler, and a lubricant in pharmaceutically compatible and pharmaceutically acceptable amounts. In one embodiment, lactose-free dosage forms comprise active ingredients, microcrystalline cellulose, pre-gelatinized starch, and magnesium stearate.

Further encompassed herein are anhydrous pharmaceutical compositions and dosage forms comprising active ingredients, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, NY, 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms provided herein can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected.

An anhydrous pharmaceutical composition should be prepared and stored such that its anhydrous nature is maintained. Accordingly, in one embodiment, provided herein are anhydrous compositions packaged using materials to prevent exposure to water such that they can be included in suitable formulary kits. Examples of suitable packaging include, but are not limited to, hermetically sealed foils, plastics, unit dose containers (e.g., vials), blister packs, and strip packs.

Encompassed herein are pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers.

In one embodiment, pharmaceutical compositions provided herein that are suitable for oral administration are formulated as discrete dosage forms, examples of which include, but are not limited to, tablets (e.g., chewable tablets), caplets, capsules, and liquids (e.g., flavored syrups). Such dosage forms contain predetermined amounts of active ingredients and may be prepared by some known methods of pharmacy. See generally, Remington's Pharmaceutical Sciences, 18th ed., Mack Publishing, Easton PA (1990).

In one embodiment, the oral dosage forms provided herein are prepared by combining the active ingredients in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

Because of their ease of administration, tablets and capsules represent the most advantageous oral dosage unit forms, in which case solid excipients are employed. If desired, tablets can be coated by standard aqueous or nonaqueous techniques. Such dosage forms may be prepared by some known methods of pharmacy. In one embodiment, pharmaceutical compositions and dosage forms are prepared by uniformly and intimately admixing the active ingredients with liquid carriers, finely divided solid carriers, or both, and then shaping the product into the desired presentation if necessary.

In one embodiment, a tablet is prepared by compression or molding. In one embodiment, compressed tablets are be prepared by compressing in a suitable machine the active ingredients in a free-flowing form, e.g., powder or granules, optionally mixed with an excipient. In one embodiment, molded tablets are made by molding in a suitable machine a mixture of a powdered compound moistened with an inert liquid diluent.

Examples of excipients that can be used in oral dosage forms provided herein include, but are not limited to, binders, fillers, disintegrants, and lubricants. Binders suitable for use in pharmaceutical compositions and dosage forms provided herein include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Suitable forms of microcrystalline cellulose include, but are not limited to, AVICEL-PH-101, AVICEL-PH-103 AVICEL RC-581, AVICEL-PH-105 (FMC Corporation, American Viscose Division, Avicel Sales, Marcus Hook, PA), and mixtures thereof. An specific binder is a mixture of microcrystalline cellulose and sodium carboxymethyl cellulose (e.g., AVICEL RC-581). Suitable anhydrous or low moisture excipients or additives include AVICEL-PH-103™ and Starch 1500 LM.

Examples of fillers suitable for use in the pharmaceutical compositions and dosage forms provided herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. In one embodiment, the binder or filler in pharmaceutical compositions provided herein is present in from about 50 to about 99 weight percent of the pharmaceutical composition or dosage form.

Disintegrants are used in the compositions provided herein to provide tablets the ability to disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms provided herein. The amount of disintegrant used varies based upon the type of formulation. In one embodiment, the pharmaceutical compositions provided herein comprise from about 0.5 to about 15 weight percent or from about 1 to about 5 weight percent of disintegrant.

Disintegrants that are suitable for use in pharmaceutical compositions and dosage forms provided herein include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums, and mixtures thereof.

Lubricants that are suitable for use in pharmaceutical compositions and dosage forms provided herein include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, but are not limited to, a syloid silica gel (AEROSIL200, W.R. Grace Co., Baltimore, MD), a coagulated aerosol of synthetic silica (Degussa Co. of Plano, TX), CAB-O-SIL (a pyrogenic silicon dioxide, Cabot Co. of Boston, MA), and mixtures thereof. In one embodiment, if used at all, lubricants are used in an amount of less than about 1 weight percent of the pharmaceutical compositions or dosage forms into which they are incorporated.

Certain pharmaceutical compositions and formulations of Compound 1 are described in U.S. Pat. No. 10,080,801, the entirety of which is incorporated herein by reference. In one embodiment, provided herein is an oral dosage form (e.g., capsule) comprising a solid form provided herein, a mixture of starch (e.g., pregelatinized starch) and lactose (e.g., anhydrous lactose), and stearic acid.

In one embodiment, provided herein is a solid oral dosage form, comprising a solid form provided herein; and one or more excipients selected from anhydrous lactose, microcrystalline cellulose, polyvinylpyrrolidone, stearic acid, colloidal anhydrous silica, and gelatin. In one embodiment, provided herein is a solid oral dosage form, comprising a solid form provided herein; and anhydrous lactose, microcrystalline cellulose, polyvinylpyrrolidone, stearic acid, colloidal anhydrous silica, and gelatin.

Depending on the state of the disease to be treated and the subject's condition, a solid form provided herein may be administered by oral, parenteral (e.g., intramuscular, intraperitoneal, intravenous, CIV, intracisternal injection or infusion, subcutaneous injection, or implant), inhalation, nasal, vaginal, rectal, sublingual, or topical (e.g., transdermal or local) routes of administration. A solid form provided herein may be formulated, alone or together, in suitable dosage unit with pharmaceutically acceptable excipients, carriers, adjuvants and vehicles, appropriate for each route of administration.

In one embodiment, a solid form provided herein is administered orally. In another embodiment, a solid form provided herein is administered parenterally. In yet another embodiment, a solid form provided herein is administered intravenously.

A solid form provided herein can be delivered as a single dose such as, e.g., a single bolus injection, or oral capsules, tablets or pills; or over time, such as, e.g., continuous infusion over time or divided bolus doses over time. A solid form provided herein can be administered repeatedly if necessary, for example, until the patient experiences stable disease or regression, or until the patient experiences disease progression or unacceptable toxicity.

A solid form provided herein can be administered once daily (QD), or divided into multiple daily doses such as twice daily (BID), three times daily (TID), and four times daily (QID). In addition, the administration can be continuous (i.e., daily for consecutive days or every day), intermittent, e.g., in cycles (i.e., including days, weeks, or months of rest without drug). As used herein, the term “daily” is intended to mean that a therapeutic compound, such as a solid form provided herein is administered once or more than once each day, for example, for a period of time. The term “continuous” is intended to mean that a therapeutic compound, such as a solid form provided herein is administered daily for an uninterrupted period of at least 7 days to 52 weeks. The term “intermittent” or “intermittently” as used herein is intended to mean stopping and starting at either regular or irregular intervals. For example, intermittent administration of a solid form provided herein is administration for one to six days per week, administration in cycles (e.g., daily administration for two to eight consecutive weeks, then a rest period with no administration for up to one week), or administration on alternate days. The term “cycling” as used herein is intended to mean that a therapeutic compound, such as a solid form provided herein is administered daily or continuously but with a rest period.

In some embodiments, the frequency of administration is in the range of about a daily dose to about a monthly dose. In one embodiment, administration is once a day, twice a day, three times a day, four times a day, once every other day, twice a week, once every week, once every two weeks, once every three weeks, or once every four weeks. In one embodiment, a solid form provided herein is administered once a day. In another embodiment, a solid form provided herein is administered twice a day. In yet another embodiment, a solid form provided herein is administered three times a day. In still another embodiment, a solid form provided herein is administered four times a day.

### Kits

In one embodiment, active ingredients provided herein are not administered to a patient at the same time or by the same route of administration. Therefore, encompassed herein are kits which, when used by the medical practitioner, can simplify the administration of appropriate amounts of active ingredients to a patient.

In one embodiment, a kit provided herein comprises a dosage form of a solid form provided herein. In one embodiment, the kits provided herein further comprise a second therapeutic agent provided herein.

In one embodiment, the kit provided herein further comprises a device that is used to administer the active ingredients. Examples of such devices include, but are not limited to, syringes, drip bags, patches, and inhalers.

In one embodiment, the kit provided herein further comprises cells or blood for transplantation as well as pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

### EXAMPLES

One embodiment of the invention are illustrated by the following non-limiting examples.

### Analytical Methods

Typical measurement conditions are provided below:

### X-Ray Powder Diffraction (XRPD)

XRPD analysis was carried out on a PANalytical X'pert pro with PIXcel detector (128 channels), scanning the samples between 3 and 35° 2θ. The material was gently ground to release any agglomerates and loaded onto a multi-well plate with Kapton or Mylar polymer film to support the sample. The multi-well plate was then placed into the diffractometer and analyzed using Cu K radiation (α₁ λ=1.54060 Å; α₂=1.54443 Å; β=1.39225 Å; α₁:α₂ ratio=0.5) running in transmission mode (step size 0.0130° 2θ, step time 18.87 s) using 40 kV/40 mA generator settings. Data were visualized and images generated using the HighScore Plus 4.7 desktop application (PANalytical, 2017).

Differential Scanning calorimetry (DSC)

Approximately 1-5 mg of material was weighed into an aluminum DSC pan and sealed non-hermetically with an aluminum lid. The sample pan was then loaded into a TA Instruments Discovery DSC 2500 differential scanning calorimeter equipped with a RCS90 cooler. The sample and reference were heated to 250 or 300° C. at a scan rate of 10° C./min and the resulting heat flow response monitored. Nitrogen was used as the cell purge gas, at a flow rate of 50 cm³/min.

### Thermogravimetric Analysis (TGA)

Approximately, 5-10 mg of material was added into a pre-tared open aluminum pan and loaded into a TA Instruments Discovery SDT 650 Auto-Simultaneous DSC and held at room temperature. The sample was then heated at a rate of 10° C./min from 30° C. to 400° C. during which time the change in sample weight was recorded. Nitrogen was used as the purge gas, at a flow rate of 200 cm³/min.

### Example 1. Preparation of Compound 1 Free Base From A

In one example, 370 mg of Compound 1 was dissolved in 18.5 ml of 1,4-dioxane. This solution was then distributed between 37 vials, 0.5 ml solution in each vial. The samples were frozen at ca. −20° C. for ca. 5 hours and dried by lyophilization overnight. 100 μl of 1,4-dioxane was added to one of the vials and the sample was heated to 40° C. on a stirrer hot plate to give a clear solution. This solution was cooled to ambient temperature and solvent was allowed to evaporate to give solids. The solids consisted of Compound 1 free base Form A.

In another example, approximately 1 g of Compound 1 was weighed into 4 vials, respectively. 60 mL of 1,4-dioxane was added to each vial and the samples were shaken in an incubator shaker at 40° C. for 2 hours to dissolve the solids. The clear solutions were allowed to cool to ambient temperature for ca. 1 hour before being stored at ca. −18° C. in a freezer. After 24 hours, the samples were defrosted at 40° C. for 1 hour before being left at ambient conditions for 1 hour and then flash frozen at −50° C. before being freeze-dried for ca. 72 hours. The solids in each sample were dissolved in 80 mL of 1,4-dioxane by shaking at 80° C. for 20 minutes. The samples were stored at −50° C. for ca. 30 minutes until completely frozen and freeze-dried for 48 hours. Approximately 500 mg of the resulting solids was weighed into a vial. 5 mL of tetrahydrofuran was added to form a slurry and the sample was temperature cycled in a shaker between ambient temperature and 40° C. with 4 hour hold at each temperature. After ca. 24 hours, the sample was filtered and dried on the filter bed for 10 minutes under vacuum before being transferred into a vial and dried at ambient temperature and pressure for ca. 17 hours. The solids consisted of Compound 1 free base Form A. The filtered mother liquor was observed to precipitate solids, which was rinsed out using a small amount of tetrahydrofuran and transferred back into its original vial. The sample was then allowed to evaporate at ambient temperature. The resulting solids consisted of Compound 1 free base Form A. A sample of Compound 1 free base Form A was characterized by XRPD (FIG. 1).

### Example 2. Preparation of Compound 1 Free Base From B

In one example, 370 mg of Compound 1 was dissolved in 18.5 ml of 1,4-dioxane. This solution was then distributed between 37 vials, 0.5 ml solution in each vial. The samples were frozen at ca. −20° C. for ca. 5 hours and dried by lyophilization overnight. 200 μl of tetrahydrofuran was added to one of the vials to generate a clear solution. The solvent was allowed to evaporate to give solids. The solids consisted of Compound 1 free base Form B.

In another example, approximately 1 g of Compound 1 was weighed into 4 vials, respectively. 60 mL of 1,4-dioxane was added to each vial and the samples were shaken in an incubator shaker at 40° C. for 2 hours to dissolve the solids. The clear solutions were allowed to cool to ambient temperature for ca. 1 hour before being stored at ca. −18° C. in a freezer. After 24 hours, the samples were defrosted at 40° C. for 1 hour before being left at ambient conditions for 1 hour and then flash frozen at −50° C. before being freeze-dried for ca. 72 hours. The solids in each sample were dissolved in 80 mL of 1,4-dioxane by shaking at 80° C. for 20 minutes. The samples were stored at −50° C. for ca. 30 minutes until completely frozen and freeze-dried for 48 hours. Approximately 500 mg of the resulting solids was weighed into a vial. 6 mL of ethanol was added to form a slurry and the sample was temperature cycled in a shaker between ambient temperature and 40° C. with 4 hour hold at each temperature. After 24 hours, a magnetic stirrer bar and 10 ml of ethanol were added to the sample. It was stirred on a hot plate at 50° C. for an hour before being allowed to cool to ambient temperature. The sample was filtered using a Buchner funnel and dried on the filter bed under suction for 20 minutes prior to transfer to a scintillation vial and dried at ambient temperature and pressure for ˜17 hours. The sample was dried further at 40° C. and ambient pressure for ˜17 hours. The solids consisted of Compound 1 free base Form B. A sample of Compound 1 free base Form B was characterized by XRPD (FIG. 2).

### Example 3. Preparation of Compound 1 Free Base From C

Approximately 2.5 g of Compound 1 was dissolved in 62.5 ml of 1,4-dioxane. This solution was distributed between 25 vials, 2.5 ml solution in each vial. The samples were frozen at ca. −18° C. in a freezer overnight. The samples were defrosted at ambient temperature and 1 ml of 1,4-dioxane was added to each vial. The samples were shaken on incubator shaker at 40° C. for 1 hour before being frozen at ca. −50° C. for 4 hours and then dried by lyophilization overnight. 600 μl of methanol:water (95:5% v/v) was added to one of the vials to give a slurry. The vial was thermally cycled between ambient temperature and 40° C. with 4 hours at each condition for ˜3 days. The solids were then isolated by centrifuge filtration and dried at ambient temperature and pressure overnight. The solids consisted of Compound 1 free base Form C. A sample of Compound 1 free base Form C was characterized by XRPD (FIG. 3).

### Example 4. Preparation of Compound 1 Free Base From D

In one example, approximately 2.5 g of Compound 1 was dissolved in 62.5 ml of 1,4-dioxane. This solution was distributed between 25 vials, 2.5 ml solution in each vial. The samples were frozen at ca. −18° C. in a freezer overnight. The samples were defrosted at ambient temperature and 1 ml of 1,4-dioxane was added to each vial. The samples were then shaken on incubator shaker at 40° C. for 1 hour before being frozen at ca. −50° C. for 4 hours and then dried by lyophilization overnight. 100 μl of acetonitrile was added to one of the vials to give a clear solution. The vial was thermally cycled between ambient and 40° C. with 4 hours at each condition for ˜3 days. The solids were then isolated by centrifuge filtration and dried at 40° C. overnight. The solids consisted of Compound 1 free base Form D.

In another example, approximately 1 g of Compound 1 was weighed into 4 vials, respectively. 60 mL of 1,4-dioxane was added to each vial and the samples were shaken in an incubator shaker at 40° C. for 2 hours to dissolve the solids. The clear solutions were allowed to cool to ambient temperature for ca. 1 hour before being stored at ca. −18° C. in a freezer. After 24 hours, the samples were defrosted at 40° C. for 1 hour before being left at ambient conditions for 1 hour and then flash frozen at −50° C. before being freeze-dried for ca. 72 hours. The solids in each sample were dissolved in 80 mL of 1,4-dioxane by shaking at 80° C. for 20 minutes. The samples were submerged in an acetone bath at −50° C. for ca. 30 minutes until completely frozen and freeze-dried for 48 hours. Approximately 500 mg of the resulting solids was weighed into a vial. 1 mL of acetonitrile was added to form a slurry and the sample was temperature cycled in an incubator shaker between ambient temperature and 40° C. with 4 hour hold at each temperature. After 24 hours, a magnetic stirrer bar and 4 ml of acetonitrile were added to the sample. It was stirred on a hot plate at ˜50° C. for an hour before being allowed to cool to ambient temperature. The sample was filtered using a Buchner funnel and dried on the filter bed under suction for 20 minutes prior to transfer to a scintillation vial and dried at ambient temperature and pressure for ca. 17 hours. The sample was dried further at 40° C. and ambient pressure for ˜17 hours. The solids consisted of Compound 1 free base Form D. A sample of Compound 1 free base Form D was characterized by XRPD (FIG. 4) and DSC (FIG. 5).

### Example 5. Preparation of Compound 1 Hydrobromide Salt From A

2.9 g of Compound 1 was dissolved in 145 mL of 1,4-dioxane. This solution was distributed between 145 vials, 1 ml solution in each vial. The samples were frozen at −20° C. for ca. 15 hours and then freeze-dried overnight for ca. 17 hours. The samples were dissolved in 1.2 mL of 1,4-dioxane and frozen at −20° C. for ca. 4 hours before being freeze-dried for ca. 21 hours. Concentrated HBr (5.5 μl, 1.1 eq.) and 300 μl tetrahydrofuran were added to one of the vials to give a slurry. The vial was then sealed and thermally cycled between ambient temperature and 40° C., with 4 hours spent under each condition. After three days, the slurry was filtered using a centrifuge filtration. The sample was dried at 40° C. under ambient pressure overnight. The solids consisted of Compound 1 hydrobromide salt From A. A sample of Compound 1 hydrobromide salt From A was characterized by XRPD (FIG. 6).

### Example 6. Preparation of Compound 1 Tosylate Salt From A

p-Toluenesulfonic acid monohydrate 236.45 mg (1.1 eq.) was added to 500 mg of Compound 1. 10 mL of ethyl acetate was added to form a slurry. The slurry was temperature cycled in an incubator shaker between ambient temperature and 40° C., with 4 hours spent under each condition, for 120 hours. The sample was isolated by filtration and dried on the filter bed for 10 minutes under vacuum before being transferred into a vial and dried at 40° C. for ca. 17 hours at ambient pressure. The solids consisted of Compound 1 tosylate salt From A. A sample of Compound 1 tosylate salt Form A was characterized by XRPD (FIG. 7), DSC (FIG. 8), and TGA (FIG. 9).

### Example 7. Preparation of Compound 1 DL-Mandelate Salt From A

2.9 g of Compound I was dissolved in 145 mL of 1,4-dioxane. This solution was distributed between 145 vials, 1 ml solution in each vial. The samples were frozen at −20° C. for ca. 15 hours and then freeze-dried overnight for ca. 17 hours. The samples were redissolved in 1.2 ml, of 1,4-dioxane and frozen at −20° C. for ca. 4 hours before being freeze-dried for ca. 21 hours. DL-mandelic acid (7.48 mg, 1.1 eq.) and 100 μl acetone were added to one of the vials to give a slurry. The vial was then sealed and thermally cycled between ambient temperature and 40° C., with 4 hours spent under each condition. After three days the slurry was filtered using a centrifuge filtration. The sample was dried at 40° C. under ambient pressure overnight. The solids consisted of Compound 1 DL-mandelate salt From A. A sample of Compound 1 DL-mandelate salt Form A was characterized by XRPD (FIG. 10).

### Example 8. Preparation of Compound 1 Glycolate Salt From A

2.9 g of Compound 1 was dissolved in 145 mL of 1,4-dioxane. This solution was distributed between 145 vials, 1 ml solution in each vial. The samples were frozen at −20° C. for ca. 15 hours and then freeze-dried overnight for ca. 17 hours. The samples were dissolved in 1.2 mL of 1,4-dioxane and frozen at −20° C. for ca. 4 hours before being freeze-dried for ca. 21 hours. Glycolic acid (3.5 mg, 1.1 eq.) and 400 μl ethyl acetate were added to one of the vials to give a slurry. The vial was then sealed and thermally cycled between ambient temperature and 40° C., with 4 hours spent under each condition. After three days the slurry was filtered using a centrifuge filtration. The sample was dried at 40° C. under ambient pressure overnight. The solids consisted of Compound 1 glycolate salt From A. A sample of Compound 1 glycolate salt Form A was characterized by XRPD (FIG. 11).

### Example 9. Preparation of Compound 1 Succinate Salt From A

2.9 g of Compound 1 was dissolved in 145 mL of 1,4-dioxane. This solution was distributed between 145 vials, 1 ml solution in each vial. The samples were frozen at −20° C. for ca. 15 hours and then freeze-dried overnight for ca. 17 hours. The samples were dissolved in 1.2 mL of 1,4-dioxane and frozen at −20° C. for ca. 4 hours before being freeze-dried for ca. 21 hours. Succinic acid (5.98 mg, 1.1 eq.) and 400 μl ethyl acetate were added to one of the vials to give a slurry. The vial was then sealed and thermally cycled between ambient temperature and 40° C., with 4 hours spent under each condition. After three days the slurry was filtered using a centrifuge filtration. The sample was dried at 40° C. under ambient pressure overnight. The solids consisted of Compound 1 succinate salt From A. A sample of Compound 1 succinate salt Form A was characterized by XRPD (FIG. 12).

### Example 10. Preparation of Compound 1 L-Lactate Salt From A

2.9 g of Compound 1 was dissolved in 145 mL of 1,4-dioxane. This solution was distributed between 145 vials, 1 ml solution in each vial. The samples were frozen at −20° C. for ca. 15 hours and then freeze-dried overnight for ca. 17 hours. The samples were dissolved in 1.2 mL of 1,4-dioxane and frozen at −20° C. for ca. 4 hours before being freeze-dried for ca. 21 hours. L-lactic acid (4.3 mg, 1.1 eq.) and 500 μl ethyl acetate were added to one of the vials to give a slurry. The vial was then sealed and thermally cycled between ambient temperature and 40° C., with 4 hours spent under each condition. After three days the slurry was filtered using a centrifuge filtration. The sample was dried at 40° C. under ambient pressure overnight. The solids consisted of Compound 1 L-lactate salt From A. A sample of Compound 1 L-lactate salt Form A was characterized by XRPD (FIG. 13).

### Example 11. Preparation of Compound 1 Citrate Salt From A

In one example, 2.9 g of Compound 1 was dissolved in 145 mL of 1,4-dioxane. This solution was distributed between 145 vials, 1 ml solution in each vial. The samples were frozen at −20° C. for ca. 15 hours and then freeze-dried overnight for ca. 17 hours. The samples were dissolved in 1.2 mL of 1,4-dioxane and frozen at −20° C. for ca. 4 hours before being freeze-dried for ca. 21 hours. Citric acid (9.47 mg, 1.1 eq.) and 300 μl tetrahydrofuran were added to one of the vials to give a slurry. The vial was then sealed and thermally cycled between ambient temperature and 40° C., with 4 hours spent under each condition. After three days the slurry was filtered using a centrifuge filtration. The sample was dried at 40° C. under ambient pressure overnight. The solids consisted of Compound 1 citrate salt From A. A sample of Compound 1 citrate salt Form A was characterized by XRPD (FIG. 14).

In another example, 500 mg of Compound I was combined with 5 mL of tetrahydrofuran to form a slurry. Citric acid (262.55 mg, 1.1 equiv.) and 2.5 mL of tetrahydrofuran were added. The slurry was thermally cycled between ambient temperature and 40° C., with 4 hours spent under each condition, for 5 days. The slurry was isolated by filtration and dried on the filter bed for 10 minutes under vacuum before being transferred into a vial and dried at 40° C. for ca. 17 hours. The solids consisted of Compound 1 citrate salt From A.

### Example 12. Preparation of Compound 2 Free Base From A

In one example, 370 mg of Compound 2 was dissolved in 40 ml of 1,4-dioxane:water (80:20 v/v). This solution was divided equally between 37 vials. The samples were frozen at ca. −10° C. and then dried via lyophilization for ca. 12 hours. The solids in one of the vials consisted of Compound 2 free base Form A. A sample of Compound 2 free base Form A was characterized by XRPD (FIG. 15).

In another example, 1 g of Compound 2 was dissolved in 40 ml of 1,4-dioxane:water (80:20 v/v) with gentle heating. The solution was frozen at −10° C. and dried by lyophilization over 48 hours. The solids consisted of Compound 2 free base Form A.

### Example 13. Preparation of Compound 2 Hydrochloride Salt From A

In one example, approximately 370 mg of HCl salt of Compound 2 was dissolved in 20 mL of water: 1,4-dioxane (75:25 v/v). The solution was evenly distributed between 37 vials, approximately 0.54 mL solution in each vial. The solutions were frozen at ca. −50° C. and then lyophilized over ca. 17 hours. 2 ml acetone was added to one of the vials and the slurry thermally cycled between ambient temperature and 40° C., with 4 hours spent under each condition. After three days the solid was isolated by centrifuge filtration. The solids consisted of Compound 2 hydrochloride salt From A.

In another example, approximately 3 g of HCl salt of Compound 2 was dissolved in 160 mL of water: 1,4-dioxane (75:25 v/v). The solution was frozen using an acetone/dry ice bath and freeze dried for ca. 22 h. 40 mL of acetone was added to approximately 1 g of the resulting solids. The sample was temperature cycled between ambient temperature and 40° C., with 4 hours spent under each condition. After 48 hours, 15 mL of acetone was added and the sample was temperature cycled for 4 days. The solids were isolated by filtration and dried under vacuum at 40° C. for ca. 18 h. The solids consisted of Compound 2 hydrochloride salt From A. A sample of Compound 2 hydrochloride salt Form A was characterized by XRPD (FIG. 16), DSC (FIG. 17), and TGA (FIG. 18).

### Example 14. Preparation of Compound 2 Hydrochloride Salt From B

In one example, approximately 1.5 g of HCl salt of Compound 2 was dissolved in 81 mL of water: 1,4-dioxane (75:25 v/v). The solution was evenly distributed between 25 vials, approximately 2.16 mL solution in each vial. The solutions were frozen at ca. −50° C. and then lyophilized over ca. 17 hours. 10 mL of water: 1,4-dioxane (75:25 v/v) was added to each vial. The solutions were frozen and lyophilized over ca. 64 hours. Water (250 μl) was added to one of the vials. The vial was sealed and thermally cycled between ambient temperature and 40° C., with 4 hours spent under each condition. After 24 hours, water (100 μl) was added and the vial was thermally cycled again. After 3 days of thermal cycling, the solids were isolated by centrifuge filtration. The solids were dried at 40° C. under ambient pressure overnight. The dried solids consisted of Compound 2 hydrochloride salt From B.

In another example, approximately 3 g of HCl salt of Compound 2 was dissolved in 160 mL of water: 1,4-dioxane (75:25 v/v). The solution was frozen using an acetone/dry ice bath and freeze dried for ca. 22 h. 8 mL of water was added to approximately 1 g of the resulting solids. The sample was temperature cycled for ca. 48 hours between ambient temperature and 40° C., with 4 hours spent under each condition. The solids were isolated by filtration and dried under vacuum at 40° C. for ca. 67 h. The solids consisted of Compound 2 hydrochloride salt From B. A sample of Compound 2 hydrochloride salt From B was characterized by XRPD (FIG. 19).

The embodiments provided herein are not to be limited in scope by the specific embodiments provided in the examples which are intended as illustrations of a few aspects of the provided embodiments and any embodiments that are functionally equivalent are encompassed by the present disclosure. Indeed, various modifications of the embodiments provided herein are in addition to those shown and described herein will become apparent to those skilled in the art and are intended to fall within the scope of the appended claims.

A number of references have been cited, the disclosures of which are incorporated herein by reference in their entirety.

What is claimed is:

1. A solid form comprising Compound 1:

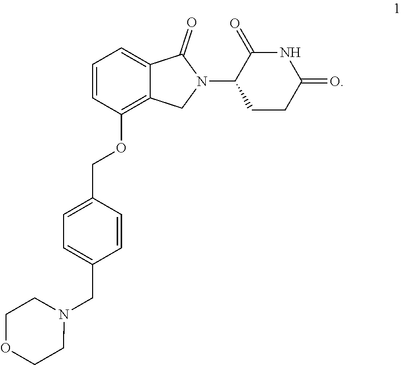 [SMILES: O=C1CC[C@H](N2Cc3c(OCc4ccc(CN5CCOCC5)cc4)cccc3C2=O)C(=O)N1]

2. The solid form of claim 1, comprising a free base of Compound 1.

3. The solid form of claim 2, which is characterized by an XRPD pattern comprising peaks at approximately 5.4, 5.7, and 6.0° 2θ.

4. The solid form of claim 3, wherein the XRPD pattern further comprises peaks at approximately 10.4, 14.4, and 20.8° 2θ.

5. The solid form of claim 4, wherein the XRPD pattern further comprises peaks at approximately 15.4, 18.2, and 19.9° 2θ.

6. The solid form of claim 3, which is characterized by an XRPD pattern that matches the XRPD pattern presented in FIG. 1.

7. The solid form of claim 2, which is characterized by an XRPD pattern comprising peaks at approximately 10.0, 11.5, and 12.5° 2θ.

8. The solid form of claim 7, wherein the XRPD pattern further comprises peaks at approximately 6.2 and 15.0° 2θ.

9. The solid form of claim 8, wherein the XRPD pattern further comprises peaks at approximately 8.8 and 16.7° 2θ.

10. The solid form of claim 7, which is characterized by an XRPD pattern that matches the XRPD pattern presented in FIG. 2.

11. The solid form of claim 2, which is characterized by an XRPD pattern comprising peaks at approximately 6.4, 7.8, and 16.3° 2θ.

12. The solid form of claim 11, wherein the XRPD pattern further comprises peaks at approximately 8.4, 13.9, and 21.3° 2θ.

13. The solid form of claim 12, wherein the XRPD pattern further comprises peaks at approximately 11.9, 12.8, and 20.2° 2θ.

14. The solid form of claim 11, which is characterized by an XRPD pattern that matches the XRPD pattern presented in FIG. 3.

15. The solid form of claim 2, which is characterized by an XRPD pattern comprising peaks at approximately 7.0, 8.2, and 17.2° 2θ.

16. The solid form of claim 15, wherein the XRPD pattern further comprises peaks at approximately 17.5 and 18.4° 2θ.

17. The solid form of claim 16, wherein the XRPD pattern further comprises peaks at approximately 4.8, 8.7, and 12.2° 2θ.

18. The solid form of claim 15, which is characterized by an XRPD pattern that matches the XRPD pattern presented in FIG. 4.

19. The solid form of claim 1, comprising a hydrobromide salt of Compound 1.

20. The solid form of claim 19, which is characterized by an XRPD pattern comprising peaks at approximately 10.0, 14.8, and 17.3° 2θ.

21. The solid form of claim 20, wherein the XRPD pattern further comprises peaks at approximately 19.1 and 22.2° 2θ.

22. The solid form of claim 21, wherein the XRPD pattern further comprises peaks at approximately 25.2 and 25.9° 2θ.

23. The solid form of claim 20, which is characterized by an XRPD pattern that matches the XRPD pattern presented in FIG. 6.

24. The solid form of claim 1, comprising a DL-mandelate salt of Compound 1.

25. The solid form of claim 24, which is characterized by an XRPD pattern comprising peaks at approximately 8.0, 13.9, and 16.5° 2θ.

26. The solid form of claim 25, wherein the XRPD pattern further comprises peaks at approximately 12.2 and 15.3° 2θ.

27. The solid form of claim 26, wherein the XRPD pattern further comprises peaks at approximately 23.9 and 24.3° 2θ.

28. The solid form of claim 25, which is characterized by an XRPD pattern that matches the XRPD pattern presented in FIG. 10.

29. The solid form of claim 1, comprising a glycolate salt of Compound 1.

30. The solid form of claim 29, which is characterized by an XRPD pattern comprising peaks at approximately 6.3, 13.9, and 16.8° 2θ.

31. The solid form of claim 30, wherein the XRPD pattern further comprises peaks at approximately 17.2 and 19.1° 2θ.

32. The solid form of claim 31, wherein the XRPD pattern further comprises peaks at approximately 22.6 and 25.4° 2θ.

33. The solid form of claim 30, which is characterized by an XRPD pattern that matches the XRPD pattern presented in FIG. 11.

34. The solid form of claim 1, comprising a succinate salt of Compound 1.

35. The solid form of claim 34, which is characterized by an XRPD pattern comprising peaks at approximately 5.6, 9.9, and 11.2° 2θ.

36. The solid form of claim 35, wherein the XRPD pattern further comprises peaks at approximately 14.5 and 16.4° 2θ.

37. The solid form of claim 36, wherein the XRPD pattern further comprises peaks at approximately 17.6 and 18.0° 2θ.

38. The solid form of claim 35, which is characterized by an XRPD pattern that matches the XRPD pattern presented in FIG. 12.

39. The solid form of claim 1, comprising a L-lactate salt of Compound 1.

40. The solid form of claim 39, which is characterized by an XRPD pattern comprising peaks at approximately 13.3, 16.0, and 18.2° 2θ.

41. The solid form of claim 40, wherein the XRPD pattern further comprises peaks at approximately 6.6 and 7.9° 2θ.

42. The solid form of claim 41, wherein the XRPD pattern further comprises peaks at approximately 12.0 and 15.6° 2θ.

43. The solid form of claim 40, which is characterized by an XRPD pattern that matches the XRPD pattern presented in FIG. 13.

44. The solid form of claim 1, comprising a citrate salt of Compound 1.

45. The solid form of claim 44, which is characterized by an XRPD pattern comprising peaks at approximately 14.0, 15.7, and 18.6° 2θ.

46. The solid form of claim 45, wherein the XRPD pattern further comprises peaks at approximately 11.6 and 17.0° 2θ.

47. The solid form of claim 46, wherein the XRPD pattern further comprises peaks at approximately 8.1 and 12.8° 2θ.

48. The solid form of claim 45, which is characterized by an XRPD pattern that matches the XRPD pattern presented in FIG. 14.

49. A solid form comprising Compound 2:

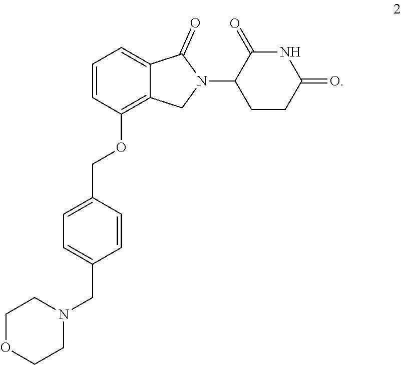 [SMILES: O=C1CCC(N2Cc3c(OCc4ccc(CN5CCOCC5)cc4)cccc3C2=O)C(=O)N1]

50. The solid form of claim 49, comprising a free base of Compound 2.

51. The solid form of claim 50, which is characterized by an XRPD pattern comprising peaks at approximately 6.3, 16.6, and 19.0° 2θ.

52. The solid form of claim 51, wherein the XRPD pattern further comprises peaks at approximately 8.6 and 13.1° 2θ.

53. The solid form of claim 52, wherein the XRPD pattern further comprises peaks at approximately 16.9 and 20.9° 2θ.

54. The solid form of claim 51, which is characterized by an XRPD pattern that matches the XRPD pattern presented in FIG. 15.

55. The solid form of claim 49, comprising a hydrochloride salt of Compound 2.

56. The solid form of claim 55, which is characterized by an XRPD pattern comprising peaks at approximately 5.0, 17.2, and 17.6° 2θ.

57. The solid form of claim 56, wherein the XRPD pattern further comprises peaks at approximately 10.4 and 14.6° 2θ.

58. The solid form of claim 57, wherein the XRPD pattern further comprises peaks at approximately 8.8 and 12.4° 2θ.

59. The solid form of claim 56, which is characterized by an XRPD pattern that matches the XRPD pattern presented in FIG. 16.

60. The solid form of claim 55, which is characterized by an XRPD pattern comprising peaks at approximately 5.9, 11.7, and 14.7° 2θ.

61. The solid form of claim 60, wherein the XRPD pattern further comprises peaks at approximately 6.7 and 12.2° 2θ.

62. The solid form of claim 61, wherein the XRPD pattern further comprises peaks at approximately 14.1 and 16.9° 2θ.

63. The solid form of claim 60, which is characterized by an XRPD pattern that matches the XRPD pattern presented in FIG. 19.

64. A pharmaceutical composition comprising a solid form of any one of claims 1 to 63, and a pharmaceutically acceptable excipient or carrier.

65. A method of treating cancer in a subject having the cancer, comprising administering to the subject a therapeutically effective amount of a solid form of any one of claims 1 to 63 or a pharmaceutical composition of claim 64.

66. The method of claim 65, wherein the cancer is lymphoma, leukemia, or myeloma.

67. The method of claim 66, wherein the cancer is multiple myeloma (MM), non-Hodgkin's lymphoma (NHL), or Hodgkin's lymphoma (HL).

68. The method of claim 65, wherein the cancer is glioblastoma.

69. The method of any one of claims 65 to 68, wherein the cancer is relapsed or refractory.

70. A method of treating an immune-related or inflammatory disease in a subject having the disease, comprising administering to the subject a therapeutically effective amount of a solid form of any one of claims 1 to 63 or a pharmaceutical composition of claim 64.

71. The method of claim 70, wherein the disease is systemic lupus erythematosus, cutaneous lupus erythematosus, or scleroderma.

72. The method of any one of claims 65 to 71, further comprising administering one or more additional active agents.

73. The method of claim 72, wherein the additional active agent is dexamethasone.

74. The method of claim 72, wherein the additional active agent is daratumumab.

75. The method of claim 72, wherein the additional active agent is a combination of daratumumab and dexamethasone.
